(19) United States
(12) Patent Application Publication
(10) Pub. No.: US 2026/0253729 A1
(43) Pub. Date: Aug. 27, 2026

(54) CLINICAL DATA ANALYSIS

(71) Applicant: SOLVENTUM INTELLECTUAL PROPERTIES COMPANUY, Maplewood, MN (US)

(72) Inventors: Jonathan D. GANDRUD, San Antonio, TX (US); Marie D. MANNER, San Antonio, TX (US); Done DEMIRGOZ, San Antonio, TX (US); Lindsey L. HINES, San Antonio, TX (US); Zane G. JOHNSON, San Antonio, TX (US)

(21) Appl. No.: 19/132,511

(22) PCT Filed: Dec. 8, 2023
(86) PCT No.: PCT/US2023/083166

§ 371 Date: May 23, 2025

Related U.S. Application Data
(60) Provisional application No. 63/432,627, filed on Dec. 14, 2022.
(60) Provisional application No. 63/460,563, filed on Apr. 19, 2023.
(60) Provisional application No. 63/462,855, filed on Apr. 28, 2023.

(51) Int. Cl. G16H 50/20

(52) U.S. Cl. CPC G16H 50/20

(57) ABSTRACT

A method for clinical data analysis includes receiving a first 3D representation that is representative of clinical data. The first three-dimensional (3D) representation includes one or more mesh elements. The method includes computing one or more mesh element features for the one or more mesh elements and providing the one or more mesh element features as an input to a first machine learning (ML) module. The method includes executing the first ML module to encode the first 3D representation into one or more latent representations and providing the one or more latent representations to a second ML module that is different from the first ML module. The method includes executing the second ML module to classify the clinical data represented in the first 3D representation into at least one predicted classification label.

### PRIORITY AND RELATED DOCUMENTS

This application is a U.S. National Stage Entry of PCT International Application No. PCT/US2023/083166, filed on Dec. 8, 2023, which claims the benefit of priority to U.S. Provisional Application No. 63/432,627, filed on Dec. 14, 2022; U.S. Provisional Application No. 63/460,563, filed on Apr. 19, 2023; U.S. Provisional Application 63/462,855, filed on Apr. 28, 2023, each of which are incorporated herein by reference.

### BACKGROUND

A proper treatment of a patient, for example, a treatment of wounds, skin, appendages, and so forth may require careful clinical analysis. For example, a state of the wound, the skin, or the appendages must be assessed before a suitable treatment can be selected and applied. In some examples, the state of the wound, the skin, or the appendages must be monitored during the treatment. As a result, there is a need for machine learning models and training approaches to improve systems that may automate the clinical analysis.

### SUMMARY

The present disclosure provides a method for clinical data analysis. The method includes receiving a first three-dimensional (3D) representation that is representative of clinical data. The first 3D representation includes one or more mesh elements. The method further includes computing one or more mesh element features for the one or more mesh elements and providing the one or more mesh element features as an input to a first machine learning (ML) module. The method further includes executing the first ML module to encode the first 3D representation into one or more latent representations and providing the one or more latent representations to a second ML module that is different from the first ML module. The method further includes executing the second ML module to classify the clinical data represented in the first 3D representation into at least one predicted classification label.

The present disclosure further provides a computing device. The computing device includes an interface configured to receive a first three-dimensional (3D) representation that is representative of clinical data. The first 3D representation includes one or more mesh elements. The computing device further includes a memory communicably coupled to the interface and configured to store the first 3D representation. The computing device further includes a processor communicably coupled to the interface and the memory. The processor is configured to compute one or more mesh element features for the one or more mesh elements; provide the one or more mesh element features as an input to a first machine learning (ML) module; execute the first ML module to encode the first 3D representation into one or more latent representations; provide the one or more latent representations to a second ML module that is different from the first ML module; and execute the second ML module to classify the clinical data represented in the first 3D representation into at least one predicted classification label.

The present disclosure further provides a method for detecting an anomaly. The method includes receiving a first three-dimensional (3D) representation that is representative of clinical data and providing the first 3D representation as an input to a first machine learning (ML) module. The method further includes executing the first ML module to encode the first 3D representation into one or more latent representations and reconstruct the one or more latent representations into a second 3D representation that is a facsimile of the first 3D representation. The method further includes computing a reconstruction error that quantifies a difference between the first 3D representation and the second 3D representation and determining at least one region of the first 3D representation that has the reconstruction error greater than a predetermined threshold. The method further includes determining that the at least one region corresponds to the anomaly.

The present disclosure further provides a method for detecting a swelling. The method includes receiving a first three-dimensional (3D) representation that is representative of clinical data. The clinical data is representative of a skin of the patient or an appendage of the patient. The method further includes providing the first 3D representation as an input to a first machine learning (ML) module and executing the first ML module to encode the first 3D representation into one or more latent representations. The method further includes providing the one or more latent representations to a second ML module that is different from the first ML module and executing the second ML module to classify the clinical data into a current state of the swelling or a future state of the swelling.

The present disclosure describes systems and techniques for training and using one or more machine learning (ML) models, such as neural networks to analyze a skin of a patient, analyze a wound on the skin of the patient, or to otherwise analyze a condition of an appendage of the patient, for the purpose of guiding a clinician in a treatment of the patient. The techniques described herein may use Representation Learning to train the neural networks to perform such analysis. Clinical data may be provided to an ML model which has been trained to perform one or more of the techniques described herein, enabling the ML model to generate an indication of a health or status of the patient which may be used by the clinician in the treatment of the patient. The clinical data may include one or more digital images (e.g., a two-dimensional (2D) raster image including a grid of pixels, such as a 2D color digital photo, a heatmap, a depth map, or a map of some other sensor-generated modality, or the like) of aspects of a body of the patient or one or more 3D representations (e.g., 3D point clouds, 3D meshes, 3D surfaces, voxelized representations, or the like) of the aspects of the body of the patient. 2D aspects of the body of the patient may include a 2D image of the wound, a dressing on the skin, the appendage, or the skin of the patient. 3D aspects of the body of the patient may include a 3D representation of the wound (e.g., a voxelized representation of an arm which may be affected by lymphedema), a 3D representation of the dressing on the skin (e.g., a 3D mesh of a dressing on top of the wound—where the dressing may have wrinkles which may lead to leaks), a 3D representation of the appendage (e.g., a 3Ds point cloud of a swollen appendage or torso), or the like. The clinician may include a physician, a nurse, a physician assistant, a technician, an emergency medical technician (EMT), a fire-fighter, a dentist, an orthodontist, a dermatologist, a chiropractor, a physical therapist, or any other practitioner who treats the patients.

In some implementations, one or more instances of the clinical data (e.g., the 2D color digital photos, or the 3D meshes) may be provided to a first ML module. The first ML module (e.g., one or more linear layers, or some portion of an encoder-decoder structure-such as an autoencoder or a transformer) may be trained to generate one or more latent representations (or latent embeddings) of the clinical data. The encoder-decoder structure may include at least one encoder and/or at least one decoder. Non-limiting examples of the encoder-decoder structure include transformers, autoencoders, such as variational autoencoders, regularized autoencoders, masked autoencoders, or capsule autoencoders. The one or more latent representations may be provided to a second ML module (e.g., a convolutional neural network, a set of fully connected layers, or some portion of the encoder-decoder structure, or the like). The second ML module may be trained to generate the indication of the health or status of the patient, based on the one or more latent representations.

In some implementations, when the 3D representation of the clinical data is provided to the first ML module, the first ML module may include at least one reconstruction autoencoder having the encoder-decoder structure. The reconstruction autoencoder is especially well suited to generate a representation (i.e., the one or more latent representations) for the clinical data, since such an autoencoder may reduce the data size of the clinical data (e.g., 3D data which may include thousands or even tens-of-thousands of mesh elements) while maintaining much of the information about a shape and/or a structure of the clinical data. This reduced-dimensionality form (e.g., a vector of 512 or 1024 real numbers, among other possible sizes) of the clinical data may occupy a latent space and/or may be more easily processed by the second ML module, due to the reduced complexity of the representation for the clinical data. The reconstruction autoencoder (e.g., a variational autoencoder optionally utilizing continuous normalizing flows) has the further advantage in that the received 3D representation of the clinical data, such as for the wound or the appendage, may be reconstructed after having been first converted into a latent form or representation (i.e., the one or more latent representations). For example, the latent representation may be reconstructed using the decoder of the reconstruction autoencoder. The reconstructed version of the 3D representation may then be compared to the original 3D representation by the computation of a reconstruction loss. A low reconstruction loss may indicate that the reconstruction autoencoder was successfully trained to encode aspects of the shape and/or the structure of the 3D representation in the latent form (e.g., a latent vector may be generated by a variational autoencoder, or a latent capsule may be generated by a capsule autoencoder), and that the latent representation of the 3D representation for the clinical data is suitable for processing by the second ML module.

Continuous normalizing flows (CNF) may include a series of invertible mappings which may transform a probability distribution. In some implementations, the CNF may be implemented by a succession of blocks in the decoder of the autoencoder. Such blocks may constrict a complex probability distribution, thereby enabling the decoder of the autoencoder to learn to map a simple distribution to a more complicated distribution and back, which leads to a data precision-related technical improvement that enables a distribution of the shapes of the 3D representation for the clinical data after reconstruction (e.g., in deployment) to be more representative of a distribution of the shapes of the 3D representation of the clinical data in a training dataset provided during a training. The invertibility of the CNF provides a technical advantage of improved mathematical efficiencies during the training, thereby providing resource usage-related technical improvements.

Aspects of the present disclosure can provide a technical solution to the technical problem of predicting, using 2D or 3D representations of 2D or 3D clinical data, a current or future state of an injury on a patient's skin, an article attached to the skin, a wound on the skin, a limb or an appendage of the patient, or an abnormality on the skin. In particular, by practicing techniques disclosed herein, computing systems specifically adapted to classify the 2D or 3D clinical data are improved. For example, aspects of the present disclosure improve the performance of the computing system for a 3D representation of clinical data by reducing consumption of computing resources. In particular, aspects of the present disclosure reduce the computing resource consumption by decimating the 3D representations of clinical data (e.g., reducing the counts of mesh elements used to describe aspects of the patient's wound, the skin, the limb, or of the article attached to the skin, etc.) so that the computing resources are not unnecessarily wasted by processing excess quantities of the mesh elements. Additionally, decimating the meshes does not reduce the overall predictive accuracy of the computing system (and indeed may actually improve predictions because an input provided to a ML model after decimation may be a more accurate (or better) representation of the 3D clinical data). For example, noise or other artifacts which are unimportant (and which may reduce an accuracy of predictive models) are removed. That is, aspects of the present disclosure provide for more efficient allocation of the computing resources and in a way that improves the accuracy of the underlying system.

Furthermore, aspects of the present disclosure may need to be executed in a time-constrained manner, such as when a wound or an abnormality on the patient's skin requires an immediate assessment while the patient waits at a clinical context or in a clinical environment. As such, aspects of the present disclosure are necessarily rooted in the underlying computer technology of the latent encoding of 2D or 3D clinical data using encoder-decoder structures (or other neural networks) and cannot be performed by a human, even with the aid of pen and paper. For instance, implementations of the present disclosure must be capable of: 1) storing thousands or millions of mesh elements in the 3D clinical data (e.g., a 3D representation of the patient's skin, limb, wound, appendage, or an article attached to the patient's body) in a manner that can be processed by a computer processor; 2) performing calculation on thousands or millions of the mesh elements in the 3D clinical data, e.g., to quantify aspects of a shape and or/a structure of the 3D representation of the clinical data; and 3) predicting, based on a machine learning model, one or more class labels to assign to the patient's body part (e.g., the patient's skin, limb, wound, appendage, or the article attached to the patient's body), and do so during the course of a short office visit.

### BRIEF DESCRIPTION OF THE DRAWINGS

Exemplary implementations disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 shows a schematic block diagram of a computing device, according to an implementation of the present disclosure;

FIG. 2 shows a flowchart of a method for clinical data analysis, according to an implementation of the present disclosure;

FIG. 3 shows a schematic block diagram of a process of the method shown in FIG. 2, according to an implementation of the present disclosure;

FIG. 4 shows a schematic block diagram of a process for training a first ML module, according to an implementation of the present disclosure;

FIG. 5 shows a schematic block diagram of a process for training a second ML module, according to an implementation of the present disclosure;

FIGS. 6A and 6B show a code implementing a 3D encoder and a 3D decoder for the first ML module, according to an implementation of the present disclosure;

FIG. 7 shows a code implementing a 2D encoder and a 2D decoder for the first ML module, according to another implementation of the present disclosure;

FIG. 8 shows a schematic block diagram of a segmentation process for segmentation of a first 3D representation of clinical data, according to an implementation of the present disclosure;

FIG. 9 shows a flowchart of a method for detecting an anomaly, according to an implementation of the present disclosure;

FIG. 10 shows a schematic block diagram of a process of the method shown in FIG. 9, according to an implementation of the present disclosure;

FIG. 11A shows different types of skin abnormalities that can be classified by the second ML module, according to an implementation of the present disclosure;

FIG. 11B shows identification of different locations of the skin abnormalities, according to an implementation of the present disclosure;

FIG. 11C shows different stages of the skin abnormalities that can be classified by the second ML module, according to an implementation of the present disclosure;

FIG. 12A shows different types of the skin abnormalities that can be classified by the second ML module, according to another implementation of the present disclosure;

FIG. 12B shows identification of different locations of the skin abnormalities, according to another implementation of the present disclosure;

FIG. 12C shows different stages of the skin abnormalities that can be classified by the second ML module, according to another implementation of the present disclosure;

FIG. 13 shows an implant site including an implant on a skin of a patient, according to an implementation of the present disclosure;

FIG. 14 shows a flowchart of a method for detecting a swelling, according to an implementation of the present disclosure;

FIG. 15 shows different states/stages of the swelling in an appendage, according to an implementation of the present disclosure;

FIG. 16 shows an article disposed on the skin of the patient, according to an implementation of the present disclosure;

FIG. 17 shows a schematic block diagram of a data augmentation process, according to an implementation of the present disclosure;

FIGS. 18 and 19 each shows an input 3D mesh and corresponding reconstructed mesh, according to an implementation of the present disclosure;

FIG. 20 shows a depiction of the reconstruction error from a reconstructed tooth, according to an implementation of the present disclosure; and

FIG. 21 shows a bar chart depicting a mean absolute distance of all vertices involved in a reconstruction of the tooth in data.

### DETAILED DESCRIPTION

In the following description, reference is made to the accompanying figures that form a part thereof and in which various implementations are shown by way of illustration. It is to be understood that other implementations are contemplated and may be made without departing from the scope or spirit of the present disclosure. The following detailed description, therefore, is not to be taken in a limiting sense.

In the following disclosure, the following definitions are adopted.

As used herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the implementations of this disclosure.

As used herein, “at least one of A and B” should be understood to mean “only A, only B, or both A and B”.

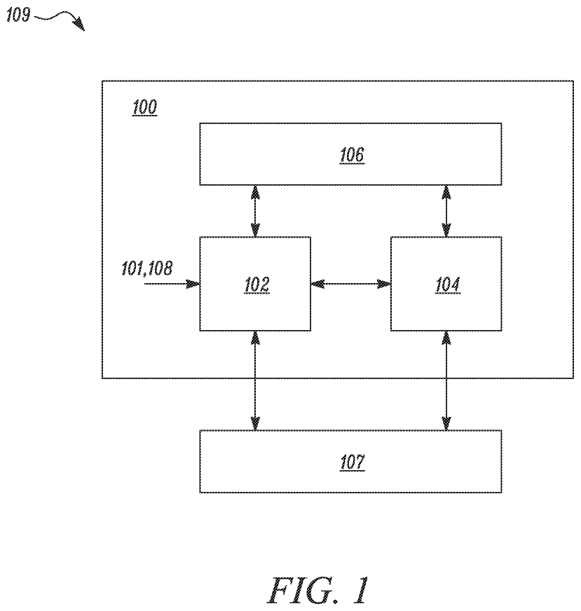

Referring now to figures, FIG. 1 is a schematic block diagram of a computing device 100, according to an implementation of the present disclosure. The computing device 100 may be used for clinical data analysis. In some implementations, the computing device 100 is deployed in a clinical environment 109.

The computing device 100 includes an interface 102. The computing device 100 further includes a memory 104 communicably coupled to the interface 102. The computing device 100 further includes a processor 106 communicably coupled to the interface 102 and the memory 104. In some implementations, the processor 106 may be interchangeably referred to as “the first processor 106”. In some implementations, the interface 102 and the memory 104 may further be communicably coupled to a second processor 107 that is different from the first processor 106. In some implementations, another device (not shown) may include the second processor 107. In some implementations, the first and second processors 106, 107 may have different data processing capabilities.

The interface 102 is configured to receive a first three-dimensional (3D) representation 108 that is representative of clinical data 101. In some implementations, the interface 102 is configured to receive a first two-dimensional (2D) representation that is representative of the clinical data 101.

The first 3D representation 108 has a different kind of data structure from that of the first 2D representation, such as a 2D image (which may include a rectilinear grid of pixels of various colors or intensities).

In some implementations, the first 3D representation 108 includes at least of a 3D point cloud, 3D surface, 3D mesh, and a voxelized representation (i.e., such as voxels used in sparse computations). In some implementations, the first 2D representation includes a 2D raster image including a grid of pixels, such as a 2D color digital photo, x-ray images, a heatmap, a depth map, or a map of some other sensor-generated modality, or the like.

The memory 104 is configured to store the first 3D representation 108. The 3D mesh may comprise edges, faces or vertices. In some implementations, the memory 104 is configured to store the first 2D representation.

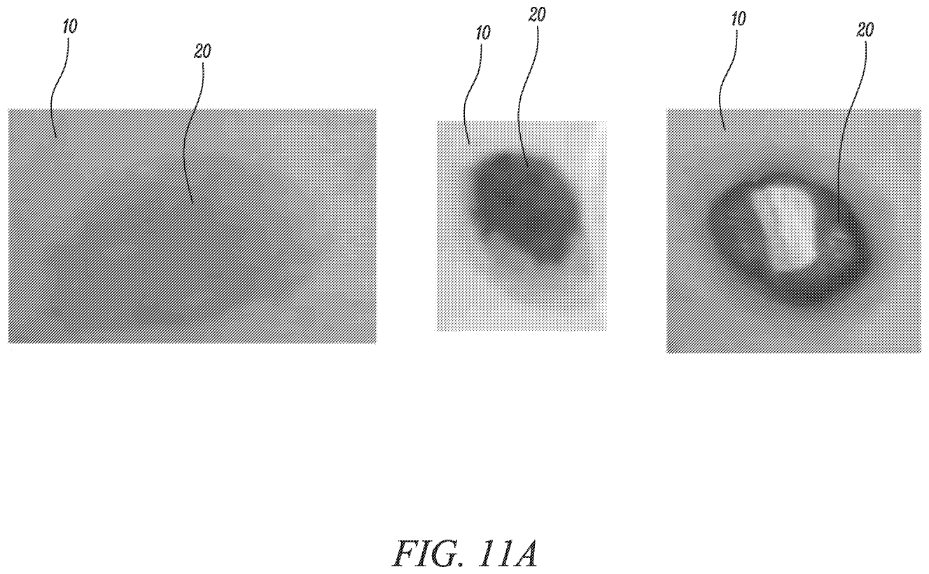

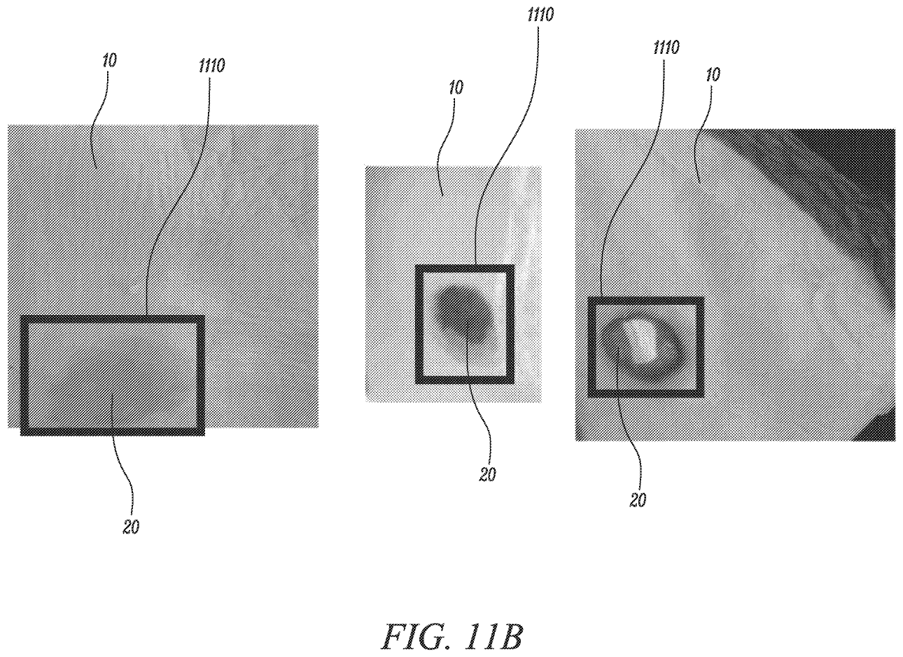

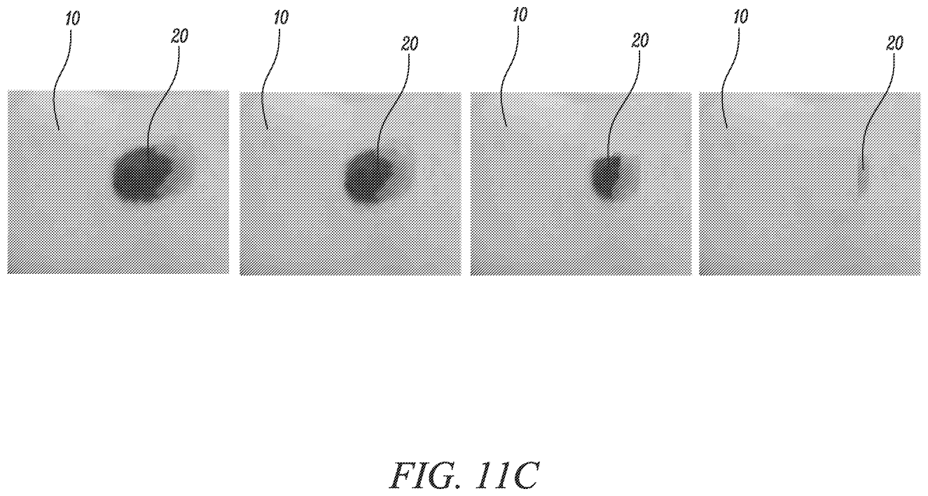

In some implementations, the clinical data 101 is representative of a skin 10 (shown in FIG. 11A) of a patient.

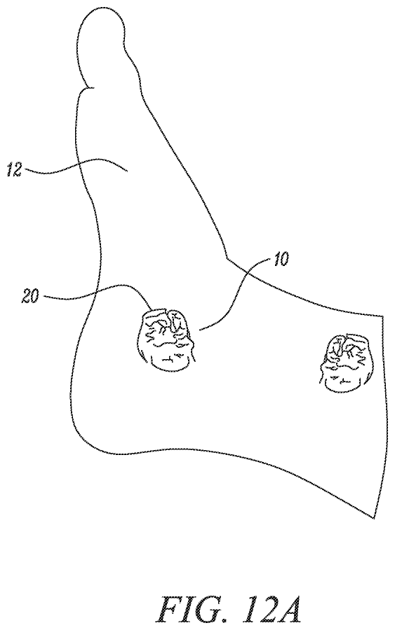

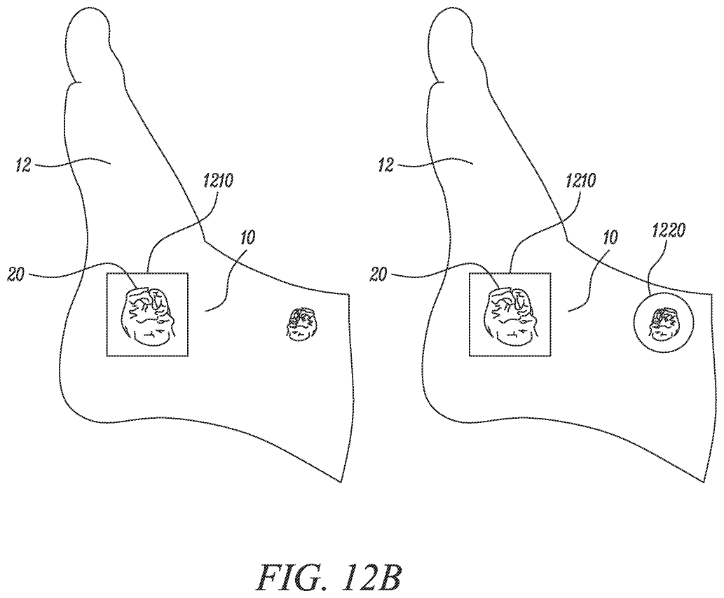

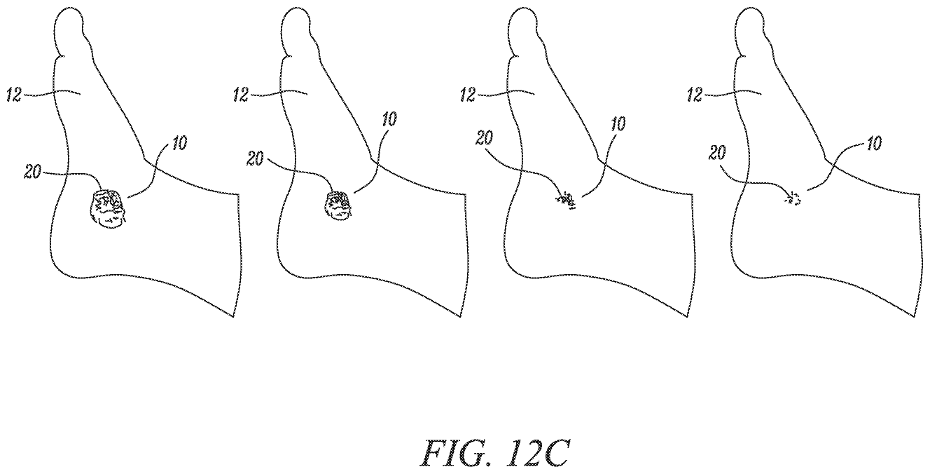

In some implementations, the clinical data 101 is representative of an appendage 12 (shown in FIG. 12A). In some implementations, the appendage 12 may include a limb (such as an arm or a leg), a hand, a foot, a digit (such as a finger or a toe), or a head of the patient. In some implementations, the clinical data 101 may be representative of a torso (not shown). In some implementations, the torso may include groin, abdomen, chest, or shoulders of the patient.

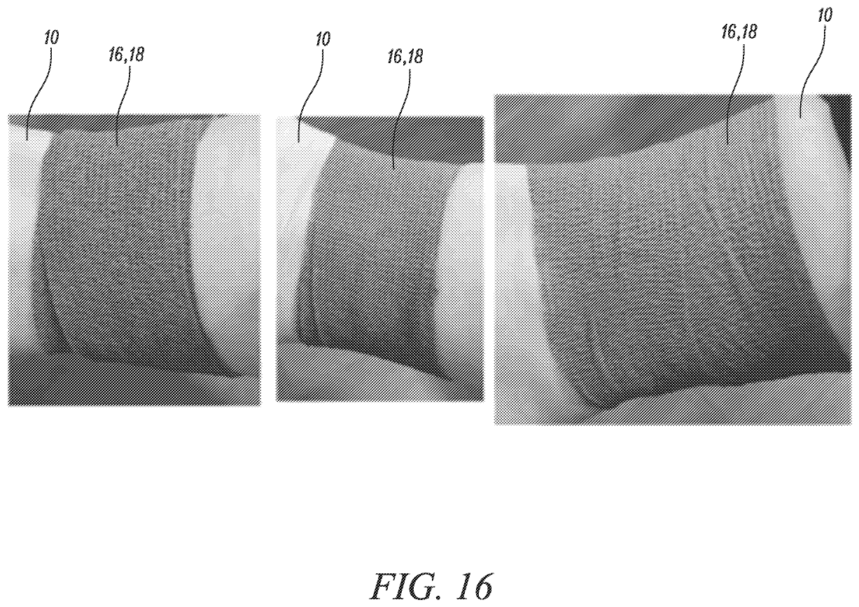

In some implementations, the clinical data 101 is representative of an article 16 (shown in FIG. 16) disposed on the skin 10 of the patient.

In some implementations, the article 16 is a wrapping 18 (shown in FIG. 16) on the skin 10 of the patient. The wrapping 18 may be, for example, a 3M Coban wrapping. In some implementations, the wrapping 18 may be a dressing. The dressing may include a bandage, a hydrocolloid dressing, a hydrogel dressing, an alginate dressing, a collagen dressing, a foam dressing, a transparent dressing (such as 3M TEGADERM products), a cloth dressing, and the like.

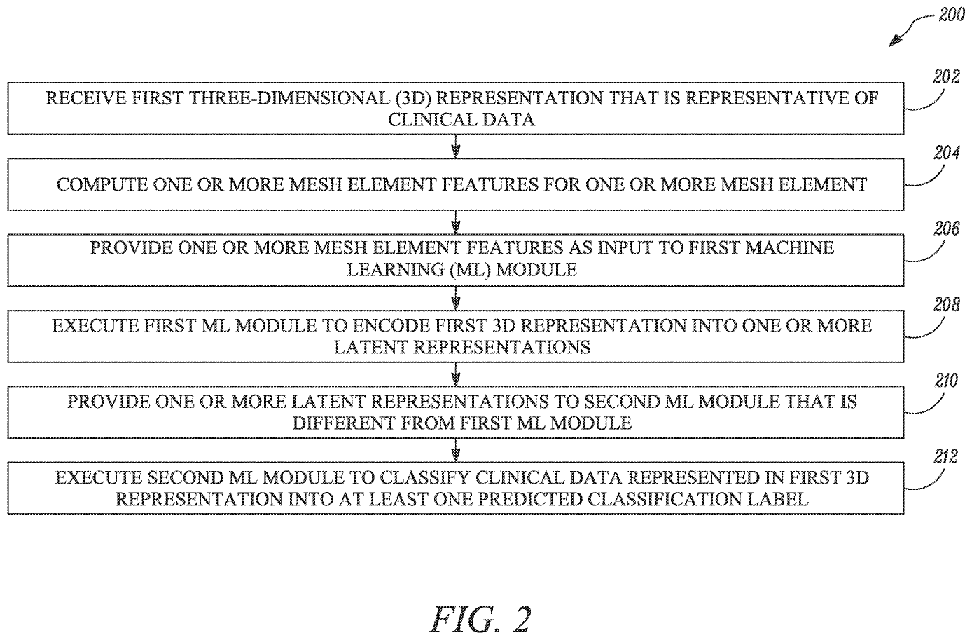

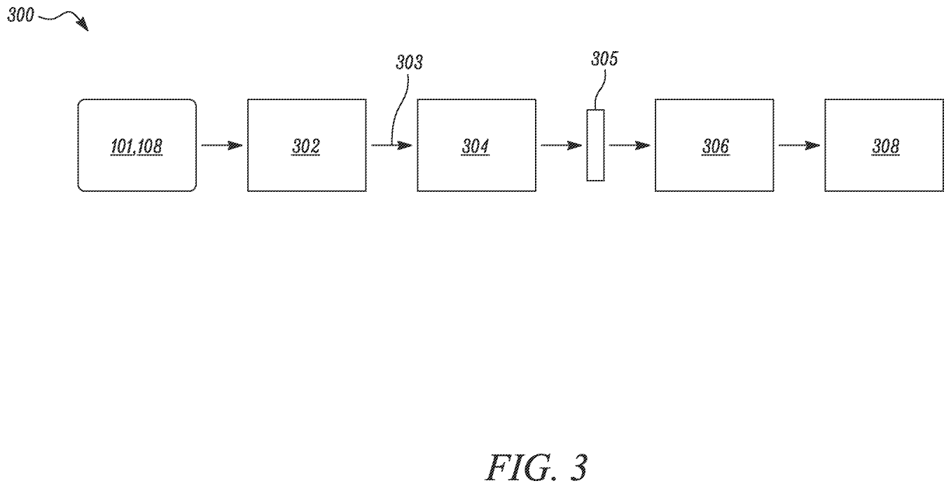

FIG. 2 is a flowchart of a method 200 for the clinical data analysis, according to an implementation of the present disclosure. FIG. 3 is a schematic block diagram of a process 300 of the method 200 shown in FIG. 2, according to an implementation of the present disclosure.

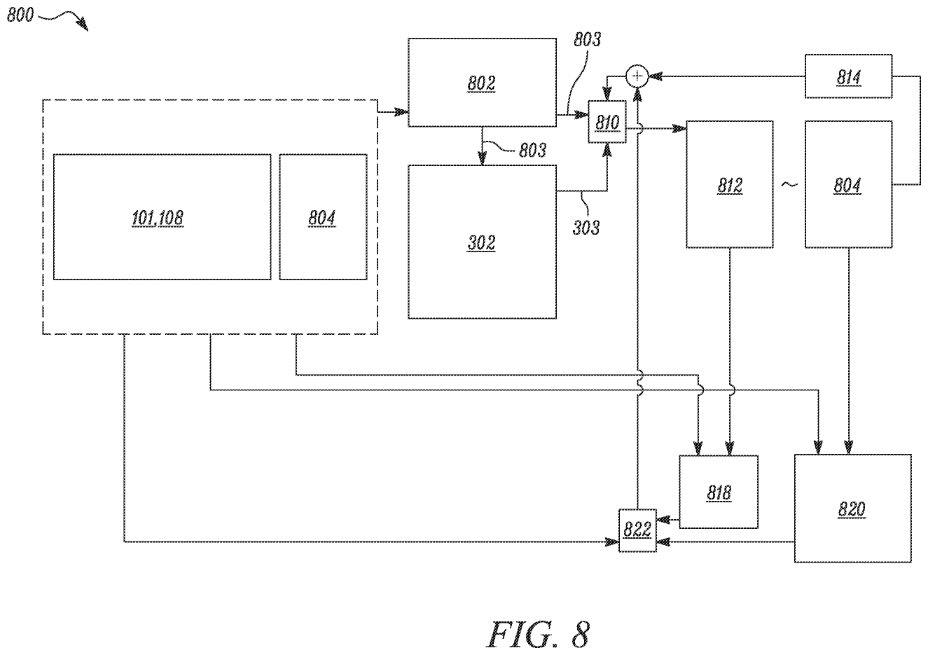

Referring to FIGS. 1 to 3, at step 202, the method 200 includes receiving the first 3D representation 108 that is representative of the clinical data 101. The first 3D representation 108 includes one or more mesh elements 803 (shown schematically in FIG. 8). Examples of the one or more mesh elements 803 may include coordinates of a vertex, or a color of the vertex, and so forth as described herein.

In some implementations, a mesh preprocessor module 802 (shown in FIG. 8) may rearrange the mesh elements 803 of the first 3D representations 108 into one or more lists of the mesh elements 803.

In some implementations, at least one of the one or more mesh elements 803 has at least one associated meta data value. In some implementations, the first 2D representation may also have at least one associated meta data value.

In some implementations, the at least one associated meta data value includes data pertaining to at least one of a color of an object, a temperature of the object, a surface impedance of the object, or other aspects of the object which can be measured using one or more sensors or one or more imaging devices. The object may be any component of the first 3D representation 108 of the clinical data 101, for example, a portion of the skin 10, the article 16, the wrapping 18, and so forth. In some implementations, the at least one associated meta data value includes data pertaining to blood oxygenation or wound oxygenation.

At step 204, the method 200 includes computing one or more mesh element features 303 for the one or more mesh elements 803.

In some implementations, the one or more mesh element features 303 may be computed by a mesh element feature module 302. In some implementations, the processor 106 is configured to compute the one or more mesh element features 303 for the one or more mesh elements 803. In some implementations, the processor 106 is configured to execute the mesh element feature module 302 to compute the one or more mesh element features 303 for the one or more mesh elements 803.

At step 206, the method 200 includes providing the one or more mesh element features 303 as an input to a first machine learning (ML) module 304. In some implementations, the first ML module 304 is an autoencoder neural network (e.g., a 3D autoencoder neural network). The first ML module 304 may further include one or more transformers, one or more fully connected layers, one or more combinations of 3D convolution and 3D pooling layers, or the like.

In some implementations, the autoencoder neural network includes a variational autoencoder (VAE) neural network. In some implementations, the autoencoder neural network includes a capsule autoencoder neural network. Additional types of the autoencoder neural networks which may be trained for use in the first ML module 304 include convolutional autoencoders (which may include U-Net convolutional models), undercomplete autoencoders, contractive autoencoders, deep belief autoencoders (such as are comprised of restricted Boltzmann machines for the encoders and decoders), sparse autoencoders, and denoising autoencoders.

An autoencoder neural network may be trained for use in a 2D domain by training the autoencoder neural network on 2D data. An autoencoder neural network may be trained for use in a 3D domain by training the autoencoder neural network on 3D data. The autoencoder neural network may improve a signal-to-noise ratio in an input data (e.g., the first 2D representation or the first 3D representation 108).

In some implementations, the processor 106 is configured to provide the one or more mesh element features 303 as the input to the first ML module 304.

In some implementations, image texture features (e.g., SIFT, SURF, ORB, BRIEF, or the like) may be computed for the first 2D representation, and subsequently be provided to the first ML module 304, along with the first 2D representation.

At step 208, the method 200 includes executing the first ML module 304 to encode the first 3D representation 108 into one or more latent representations 305 (or one or more latent embeddings). Therefore, the first ML module 304 may be a Representation Generation Module. The one or more latent representations 305 may be information-rich and reduced-dimensionality representations of the first 3D representation 108 of the clinical data 101. For example, the one or more latent representations 305 may include a latent vector or a latent capsule.

Furthermore, in some implementations, either of a transformer encoder or a transformer decoder may generate the one or more latent representations 305, which may be outputted by the first ML module 304. In some implementations, an encoder portion of the variational autoencoder may generate the one or more latent representations 305. In some implementations, a capsule encoder portion of the capsule autoencoder may generate the one or more latent representations 305.

As discussed above, in some implementations, the processor 106 is configured to provide the one or more mesh element features 303 as the input to the first ML module 304. The mesh element features 303 may improve the ability of the first ML module 304 to encode a shape and/or a structure of the clinical data 101 into the latent form.

In some implementations, the processor 106 is configured to execute the first ML module 304 to encode the first 3D representation 108 into the one or more latent representations 305. Therefore, in some implementations, executing the first ML module 304 to encode the first 3D representation 108 into the one or more latent representations 305 includes executing the first ML module 304, by the first processor 106, to encode the first 3D representation 108 into the one or more latent representations 305.

At step 210, the method 200 includes providing the one or more latent representations 305 to a second ML module 306 that is different from the first ML module 304. In some implementations, the second ML module 306 may include a neural network (e.g., a convolutional neural network, a set of fully connected layers, or some portion of the encoder-decoder structure, or the like), or a non-neural network ML model (e.g., a support vector machine (SVM) model, a logistic regression model, or other ML model described herein).

In some implementations, the second ML module 306 may include a multi-layer perceptron (MLP) (e.g., 2, 3, 4, or more fully connected layers, with optional skip connections), a transformer, an autoencoder, a decision tree(s), a K-nearest neighbor model, a Naïve Bayes model, a Random Forest model, a gradient boosting model, or others described herein.

In some implementations, the processor 106 is configured to provide the one or more latent representations 305 to the second ML module 306.

At step 212, the method 200 includes executing the second ML module 306 to classify the clinical data 101 represented in the first 3D representation 108 into at least one predicted classification label 308. In some implementations, the method 200 includes executing the second ML module 306 to classify the one or more latent representations 305 into the at least one predicted classification label 308.

In some implementations, the processor 106 is configured to execute the second ML module 306 to classify the clinical data 101 represented in the first 3D representation 108 into the at least one predicted classification label 308. Therefore, in some implementations, executing the second ML module 306 to classify the clinical data 101 represented in the first 3D representation 108 into the at least one predicted classification label 308 includes executing, by the first processor 106, to classify the clinical data 101 represented in the first 3D representation 108 into the at least one predicted classification label 308.

However, in some other implementations, executing the second ML module 306 to classify the clinical data 101 represented in the first 3D representation 108 into the at least one predicted classification label 308 includes executing, by the second processor 107 that is different from the first processor 106, to classify the clinical data 101 represented in the first 3D representation 108 into the at least one predicted classification label 308.

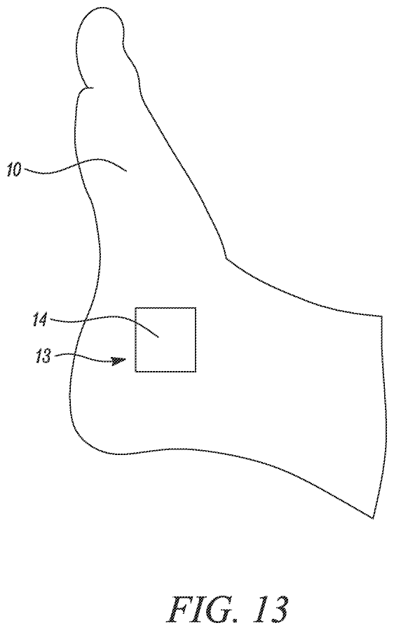

In some implementations, the second ML module 306 is configured to classify the clinical data 101 into at least one of a type of a skin abnormality 20 (shown in FIG. 11A), a current state of the skin abnormality 20, a future state of the skin abnormality 20, a current state of an implant site 13 (shown in FIG. 13) including an implant 14 (shown in FIG. 13) on the skin 10, and a future state of the implant site 13 on the skin 10.

In some implementations, the type of the skin abnormality 20 is at least one of a tumor, a wound, a burn, a rash, a puncture, a cyst, an infection, a skin growth, a bruise, a cut, a tear, an abrasion, a scratch, an ulcer, and a laceration. The skin abnormality 20 may further include a gash or a scrape.

In some implementations, the implant 14 is at least one of a skin graft (e.g., a tissue graft) and a device implant (e.g., a prosthetic).

The implant 14 may include a decorative implant, a medical implant (e.g., portacaths or subcutaneous ports), a ventricular assist device, a near field communication (NFC) chip-such as to use for making an electronic payment, a microchip, a wireless key (such as to unlock a car), a radio-frequency identification (RFID) tag, a device that monitors bodily vitals, such as a blood flow tracking, a temperature or a heart rate, an automatic blood sugar monitoring, or a regulating device, a device which vibrates in response to environmental conditions (e.g., turning to face due North), etc.

The skin graft may include a regenerative cell therapy, a limb reattachment, a skin reattachment, replacing skin flaps (e.g., from degloving injuries), a bone reconstruction, an internal organ transplant, etc.

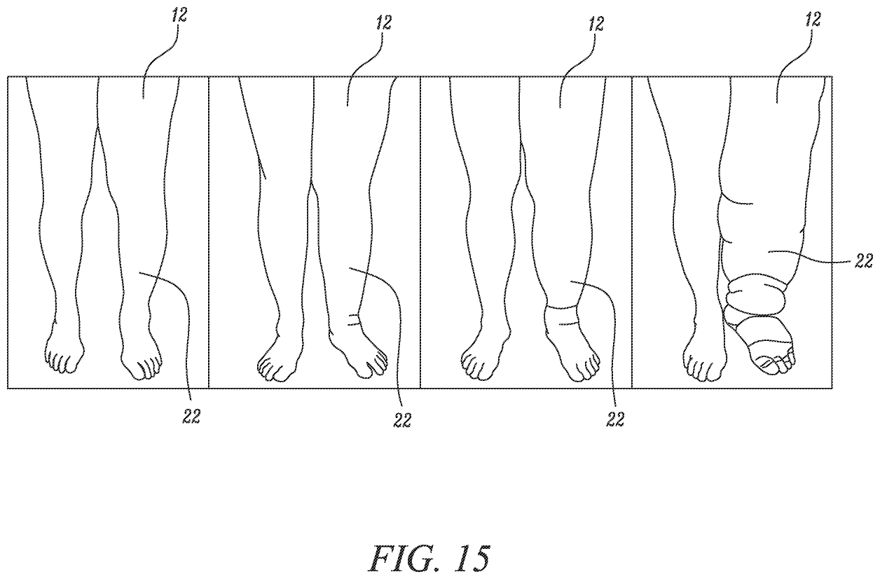

In some implementations, the second ML module 306 is configured to classify the clinical data 101 into a current state of a swelling 22 (shown in FIG. 15) in the appendage 12 or a future state of the swelling 22 in the appendage 12. In some implementations, the second ML module 306 is configured to classify the clinical data 101 into a current state of a swelling in the torso or a future state of the swelling in the torso.

In some implementations, the second ML module 306 is configured to classify the clinical data 101 into a current state of the article 16 (shown in FIG. 16). In some implementations, the current state of the article 16 includes a fit of the wrapping 18 (shown in FIG. 16) on the skin 10 of the patient.

In some implementations, the first ML module 304 may be a 2D autoencoder neural network. In such implementations, the first 2D representation may be provided as the input to the first ML module 304. In some implementations, the first ML module 304 may encode the first 2D representation into the one or more latent representations 305. In such implementations, the second ML module 306 may be executed to classify the clinical data 101 represented in the first 2D representation into the at least one predicted classification label 308.

The at least one predicted classification label 308 may pertain to a body part (such as the appendage, the torso, or the skin), the articles (e.g., the article 16, the implant 14, etc.) attached to the body part, and/or a state of health of the patient. The at least one predicted classification label 308 may be subsequently used by the clinicians in the treatment of the patient. For example, the at least one predicted classification label 308 may be used to make treatment decisions for the patient, or to recommend a treatment for the patient. In some instances, the process 300 may be enabled to operate in deployment on a handheld device (e.g., the computing device 100) for use in the clinical environment 109.

The first and second ML modules 304, 306 may be deployed in a cloud computing environment, on a mobile device, on a laptop, on a desktop computer, in an augmented reality headset, or on another computing device.

In some implementations, outputs generated by either or both of the first and second ML modules 304, 306 may be sent to the clinicians via a notification (e.g., over SMS, email, or other electronic means). In some implementations, a visualization may be made of a portion of the body part (e.g., pertaining to the clinical data 101), where graphics or other indicia are interposed on the visualization to highlight the output of the second ML module 306. In some instances, the visualization may be shown to the clinician via the mobile device, the smart phone, the laptop, or the desktop computer. The treatment may then be rendered to the patient by the clinician, as a result of inspecting the output of the second ML module 306.

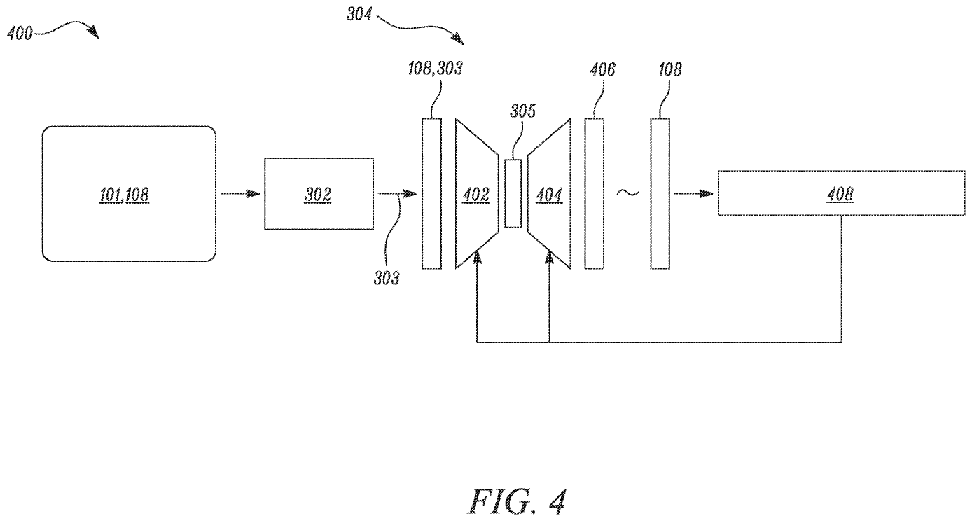

FIG. 4 is a schematic block diagram of a process 400 for training the first ML module 304, according to an implementation of the present disclosure.

For example, when the first ML module 304 includes one or more of the autoencoder neural network, the one or more of the autoencoder neural network may be trained on either 2D representations or 3D representations of the clinical data 101, such as the skin 10 (including healthy skin, the wounds, the incisions, the burns, the rashes, the skin growths, the tissue grafts, the bruises, and the like), and/or health care materials in association with skin, such the articles 16 (including the dressings, casts, splints, compression garments, and the like).

Referring to FIGS. 1, 2, and 4, in some implementations, the method 200 further includes executing the first ML module 304 to reconstruct the one or more latent representations 305 into a second 3D representation 406 that is a facsimile of the first 3D representation 108. In some implementations, the processor 106 is further configured to execute the first ML module 304 to reconstruct the one or more latent representations 305 into the second 3D representation 406 that is the facsimile of the first 3D representation 108.

In some implementations, when the first ML module 304 may encode the first 2D representation into the one or more latent representations 305, the first ML module 304 may be executed to reconstruct the one or more latent representations 305 into a second 2D representation that is a facsimile of the first 2D representation.

In some implementations, the first ML module 304 has an encoder-decoder structure including one or more encoders, or one or more decoders. Examples of the encoder-decoder structure include U-Nets, autoencoders, pyramid encoder-decoders, transformers, and so forth. In some implementations, the first ML module 304 has one or more sets of 3D convolution and 3D pooling layers.

As discussed above, in some implementations, the first ML module 304 is the autoencoder neural network including an encoder 402 and a decoder 404 (e.g., the 3D autoencoder neural network having a 3D encoder and a 3D decoder or the 2D autoencoder neural network having a 2D encoder and a 2D decoder). Specifically, in some implementations, the 3D encoder of the 3D autoencoder neural network is configured to encode the first 3D representation 108 into the one or more latent representations 305 and the 3D decoder of the 3D autoencoder neural network is configured to reconstruct the one or more latent representations 305 into the second 3D representation 406. Similarly, in some implementations, the 2D encoder of the 2D autoencoder neural network is configured to encode the first 2D representation into the one or more latent representations 305 and the 2D decoder of the 2D autoencoder neural network is configured to reconstruct the one or more latent representations 305 into the second 2D representation.

In such implementations, the first ML module 304 may further be used to improve a security or a transmission speed of the clinical data 101.

For example, the first 2D or 3D representations of the clinical data 101 may be collected by the clinicians, and then undergo subsequent encoding using the 2D or 3D encoder of the first ML module 304 to generate the one or more latent representations 305 for each example of the clinical data 101 which is provided to the first ML module 304. For example, a 2D image of a wound may be generated by a patient at a remote site (e.g., while hiking), and subsequently be provided to the first ML module 304 (e.g., which may be running locally), generating the one or more latent representations 305. When the second ML module 306 is located in a cloud server or on another remote server, the one or more latent representations 305 may be uploaded to the remote server for subsequent classification by the second ML module 306 (e.g., via the second processor 107 shown in FIG. 1). The one or more latent representations 305 may be a compressed or reduced dimensionally version of the original clinical data 101, which may reduce a bandwidth required for transmission.

In some implementations, the decoder 404 of the autoencoder neural network is remotely located from the encoder 402. The decoder 404 may be located on the remote server and be used to reconstruct the one or more latent representations 305 of the clinical data 101 to generate, for example, the second 2D representation (e.g., a reconstructed photo of the wound). In some implementations, the reconstructed photo of the wound may be inspected by the clinicians, in order to make treatment decisions for the patient. This conversion of the clinical data 101 to the one or more latent representations 305 and subsequent transmission of the one or more latent representations 305 may preserve anonymity and confidentiality of the patient.

In some implementations, the method 200 further includes computing a reconstruction loss 408 that quantifies a difference between the first 3D representation 108 and the second 3D representation 406. In some implementations, the method 200 further includes using the reconstruction loss 408 to train the first ML module 304. In some implementations, the method 200 further includes using the reconstruction loss 408 to train the first ML module 304 using backpropagation. In some implementations, using the reconstruction loss 408 to train the first ML module 304 includes providing the reconstruction loss 408 to at least one of the encoder 402 and the decoder 404 to train the first ML module 304.

In some implementations, a reconstruction loss may quantify a difference between the first 2D representation and the second 2D representation.

In an example, mesh element labeling operations may be applied on the clinical data 101 (e.g., mesh segmentation to isolate the wound, or mesh cleanup to remove extraneous material from the 3D mesh-such as 3D scanning artifacts). In some implementations, a registration step to align (e.g., using an iterative closest point technique, or the like) may be performed on the clinical data 101 with a template mesh (e.g., a template of the appendage 12 or other anatomical object). This may provide technical enhancement of improving an accuracy and a data precision of mesh correspondence computation. Further, correspondences between an example of the 3D mesh of the clinical data 101 and the corresponding template mesh may be computed, with the technical improvement of conditioning the clinical data 101 to be ready to be provided to the reconstruction autoencoder, i.e., the first ML module 304. The dataset of the prepared clinical data examples may be split into train, validation, and holdout test sets, which may then be used to train the reconstruction autoencoder. The reconstruction autoencoder may be trained using a combination of reconstruction loss and KL-Divergence loss, and optionally other examples of loss functions described herein.

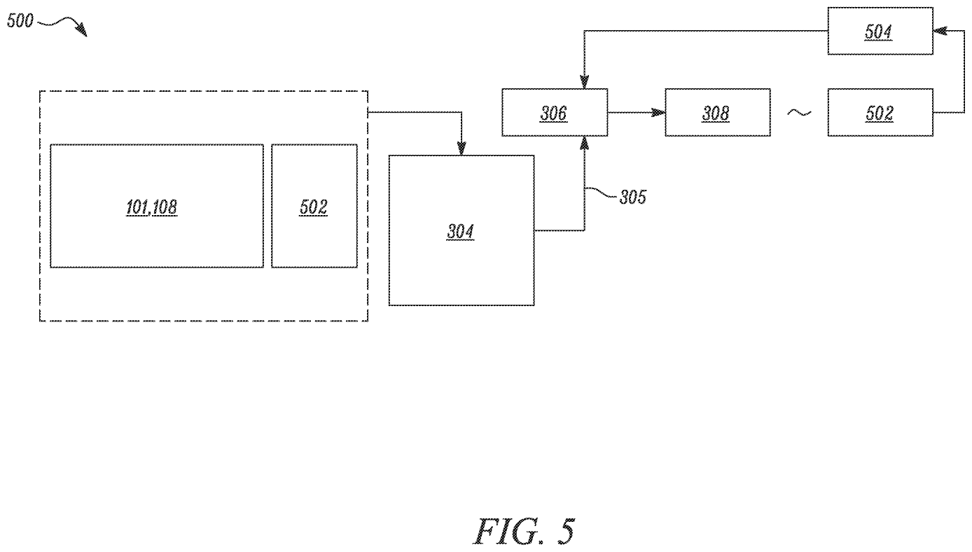

FIG. 5 is a schematic block diagram of a process 500 for training the second ML module 306, according to an implementation of the present disclosure.

Referring to FIGS. 2 and 5, in some implementations, the method 200 further includes receiving at least one ground truth classification label 502 for the first 3D representation 108. In some implementations, at least one ground truth classification label for the first 2D representation may be received. The ground truth classification labels 502 may be provided by an authority which is known to be correct.

In some implementations, the method 200 further includes computing a loss 504 that quantifies a difference between the at least one ground truth classification label 502 and the at least one predicted classification label 308. In some implementations, the loss 504 may quantify a difference between the at least one ground truth classification label for the first 2D representation and the at least one predicted classification label 308 for the first 2D representation.

In some implementations, the method 200 further includes using the loss 504 to train the second ML module 306. In some implementations, the method 200 further includes using the loss 504 to train the second ML module 306 using backpropagation.

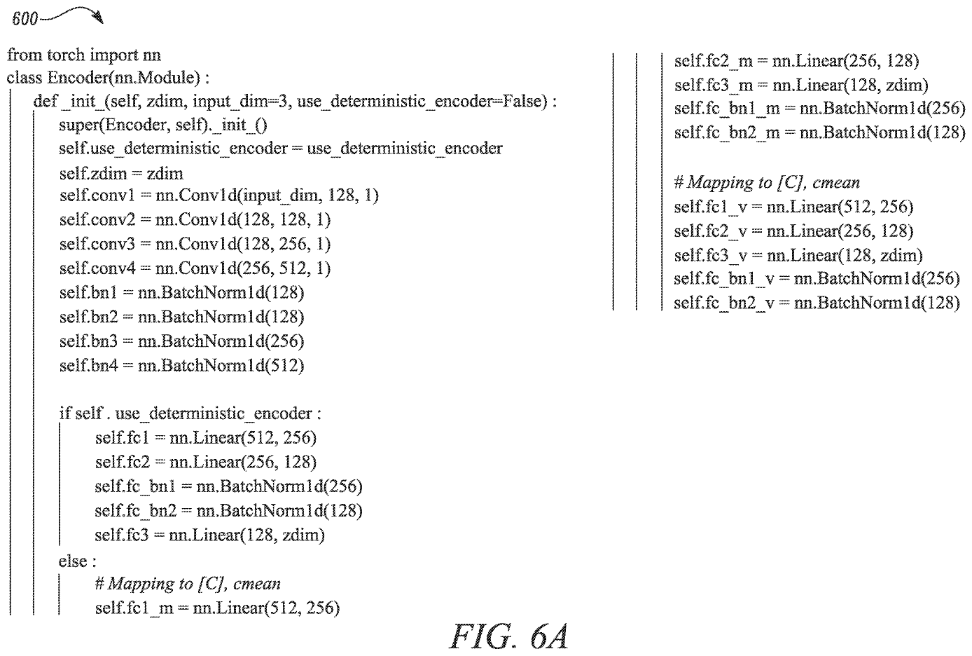

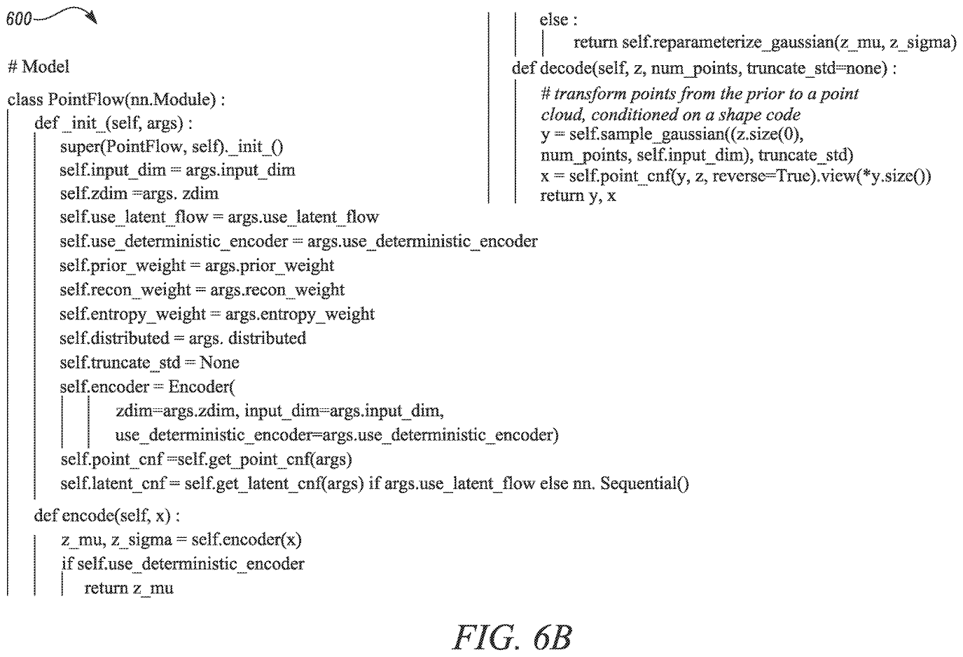

FIGS. 6A and 6B show a code 600 implementing the 3D encoder (e.g., the encoder 402 shown in FIG. 4) and the 3D decoder (e.g., the decoder 404 shown in FIG. 4) for the first ML module 304, according to an implementation of the present disclosure. Specifically, in FIGS. 6A and 6B, the code 600 is for the 3D autoencoder neural network.

These implementations may include: convolution layers, batch norm layers, linear neural network layers, Gaussian operations, and continuous normalizing flows (CNF), among others.

One of the steps which may take place in the VAE training data pre-processing is the calculation of mesh correspondences. The mesh correspondences may be computed between the mesh elements of an input mesh and the mesh elements of a reference or template mesh with known structure (e.g., a template representation). The template representation may include one or more mesh elements which are arranged in a standardized order (e.g., in manner that is consistent with an arrangement that was used in training the autoencoder neural network). In deployment, a trial 3D representation (e.g., a mesh of a pre-restoration tooth mesh of a patient, an appliance component which is to undergo modification, or a fixture model which is to undergo modification) may undergo correspondence calculation, to compute one or more correspondences between the trial 3D representation and a corresponding template representation. These correspondences enable the mesh elements of the trial 3D representation to be rearranged into an ordering which is consistent with the arrangements of mesh elements of training examples that were used in training the autoencoder neural network. This leads to improved autoencoder reconstruction accuracy, due to an improvement in the signal to noise ratio.

Stated another way, the aim of the mesh correspondence calculation is to compute correspondences between the mesh elements of the surfaces of a trial input mesh and a template (reference) mesh (e.g., a template representation). The mesh correspondence may generate point to point correspondences between the trial input and template meshes by mapping each vertex from the trial input mesh to at least one vertex in the template mesh. The correspondences may be computed between the mesh elements of the trial input mesh and the mesh elements of the reference or template mesh with known or pre-confirmed structure.

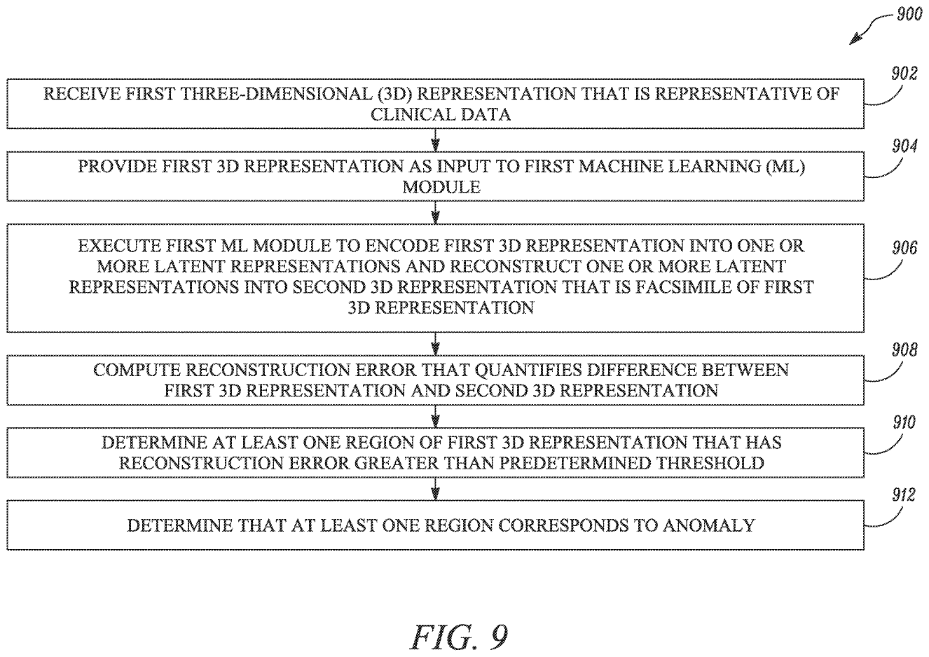

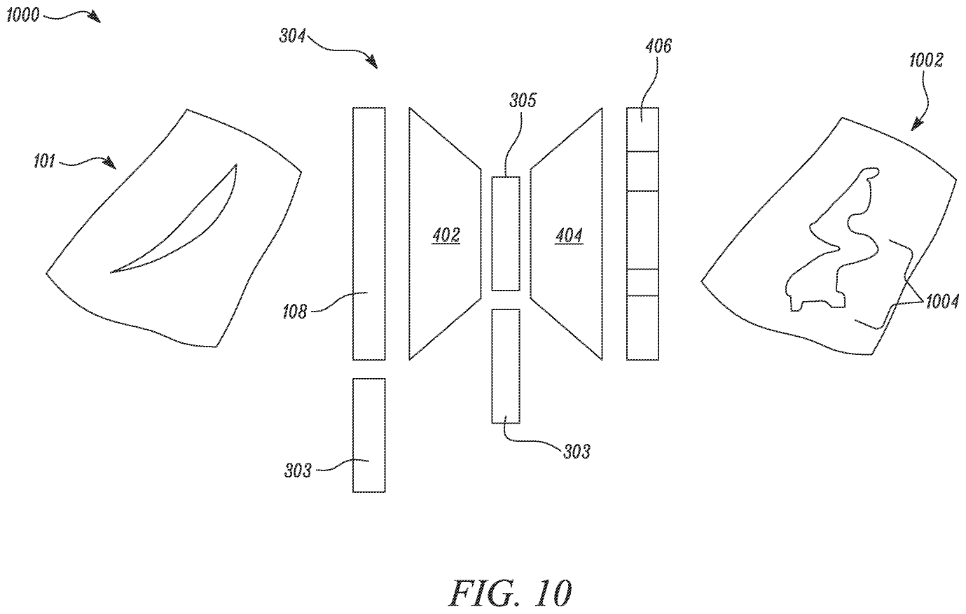

Use of the mesh correspondences may provide the data precision improvement in the mesh reconstruction, because the mesh correspondence may reduce sampling error by the encoder 402, improve alignment, and improve mesh generation quality. In some implementations, an iterative closest point (ICP) algorithm may be run between the clinical data 101 and a corresponding 3D template, during the computation of the mesh correspondences. The mesh correspondences may be computed to establish vertex-to-vertex relationships, for use in computing a reconstruction error (as described herein with respect to FIGS. 9 and 10).

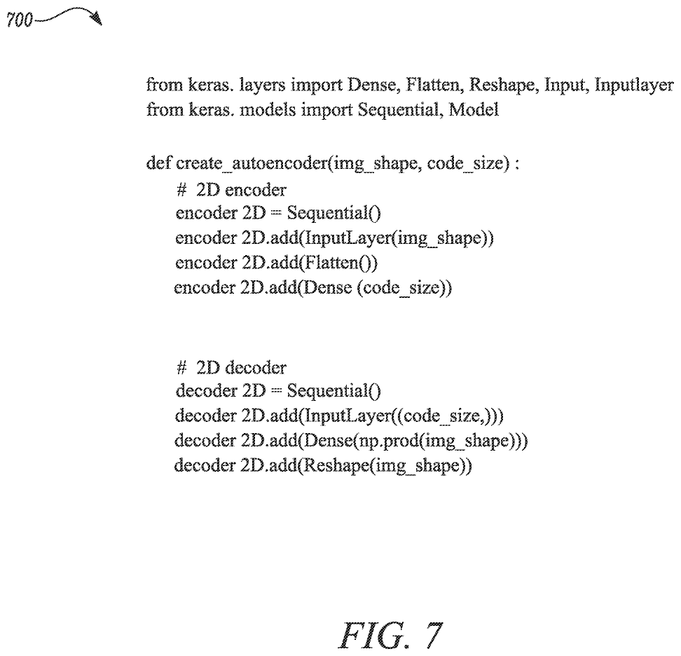

FIG. 7 shows a code 700 implementing the 2D encoder (e.g., the encoder 402 shown in FIG. 4) and the 2D decoder (e.g., the decoder 404 shown in FIG. 4) for the first ML module 304, according to an implementation of the present disclosure. Specifically, in FIG. 7, the code 700 is for the 2D autoencoder neural network.

FIG. 8 is a schematic block diagram of a segmentation process 800 for segmentation of the first 3D representation 108 of the clinical data 101, according to an implementation of the present disclosure.

In some implementations, geometric deep learning (GDL) techniques of segmenting 3D representations, such as 3D mesh segmentation, may be applied to segment of the first 3D representation 108 using a generative adversarial network (GAN). The segmentation process 800 further shows use of the GAN to train a neural network to segment the first 3D representation 108. The techniques of segmenting 3D representations may be applied to the first 3D representation 108 of the skin 10, the appendage, the torso, the skin abnormalities 20, etc.

The techniques of segmenting the 3D representations may further be applied to the first 3D representation 108 including objects, such as the article 16 or the implant 14. This may be to localize those objects or to facilitate removal of those objects from the first 3D representation 108.

The first 3D representations 108 of the clinical data 101 and corresponding ground truth inputs 804 (i.e., ground truth mesh element labels) are provided to the segmentation process 800.

As discussed above, in some implementations, the mesh preprocessor module 802 may convert the first 3D representations 108 into the one or more mesh elements 803. Further, the mesh element feature module 302 may compute the one or more mesh element features 303 for the one or more mesh elements 803.

In some implementations, the first 3D representation 108 may be provided to the mesh element feature module 302, to compute the one or more mesh element features 303 for the one or more mesh elements 803. The output (i.e., the one or more mesh element features 303) of the mesh element feature module 302 may be provided to a generator 810.

Although the generator 810 may benefit from training that includes a discriminator 822, the generator 810 may alternatively be trained without the discriminator 822. The generator 810 receives an input (e.g., the one or more mesh elements 803 and the one or more mesh element features 303). The generator 810 uses the received input to determine predicted outputs 812 pertaining to the first 3D representation 108, according to particular implementations. For instance, for segmentation, the generator 810 may be configured to predict mesh element labels for use in segmentation or mesh cleanup.

A segmented output of the segmentation process 800, i.e., the predicted outputs 812 may include mesh element labels for the one or more mesh elements 803 (e.g., one or more lists of mesh elements).

The ground truth inputs 804 may describe verified or otherwise known to be accurate labels for the one or more mesh elements 803 (e.g., the ground truth mesh element labels “correct” and “incorrect”) related to the segmented outputs performed on the first 3D representations 108. According to particular implementations, the mesh element labels described in relation to segmentation operations (or mesh cleanup operations) can be used to specify a particular collection of the one or more mesh elements 803 (such as a “point” element, an “edge” element, a “face” element, a “vertex” element, a “voxel” element, or the like) for a particular aspect of the first 3D representation 108 of the clinical data 101. For instance, a single triangle polygon of the 3D mesh includes 3 edge elements, 3 vertex elements, and 1 face element. Therefore, it should be appreciated that a segmented 3D representation consisting of many polygons can have a large number of labels associated with the first 3D representation 108.

A difference between the predicted outputs 812 and the ground truth inputs 804 can be used to compute one or more loss values 814. For instance, the loss values 814 can represent a regression loss between the predicted outputs 812 and the ground truth inputs 804. That is, according to one implementation, the loss values 814 reflect a percentage by which the predicted outputs 812 deviate from the ground truth inputs 804. The loss values 814 may include an L2 loss, a smooth L1 loss, or some other kind of loss. According to particular implementations, the L1 loss is defined as:

$$L1 = {\sum }_{i = 0}^{n} \vert {P}_{i} - {G}_{i} \vert$$

Further, according to particular implementations, the L2 loss can be defined as:

$$L2 = {\sum }_{i = 0}^{n} {( {P}_{i} - {G}_{i} )}^{2}$$

where P represents the predicted outputs 812 and G represents the ground truth inputs 804.

In addition, and as will be described in more detail below, the loss values 814 can be provided to the generator 810 to further train the generator 810, e.g., by modifying one or more weights in a neural network of the generator 810 to train the underlying model and improve ability to generate the predicted outputs 812 that mirror or substantially mirror the ground truth inputs 804.

Any of these losses (i.e., the L1 and L2 losses) can be used to supply a loss value (i.e., the loss values 814) for use in training the neural network of the generator 810 by way of a suitable training algorithm, such as backpropagation. In some instances, an accuracy score may be used in the training of the neural network. The accuracy score may quantify the difference between a data structure of the predicted output 812 and a data structure of the ground truth input 804. The accuracy score (e.g., in normalized form) may be fed back into the neural network in the course of training the neural network, for example, through backpropagation.

In the case of segmentation, the accuracy score may count matching mesh labels between a predicted mesh and a ground truth mesh (i.e., where each mesh element has an associated label). The higher the percentage of the matching mesh labels, the better the prediction (i.e., when comparing predicted labels, i.e., the predicted outputs 812 to ground truth labels, i.e., the ground truth inputs 804).

A similar accuracy score may be computed in the case of the mesh cleanup, which also predicts labels for the mesh elements. The mesh cleanup may, in some implementations, perform operations of the labeled mesh elements, such as transforming or removing the mesh elements.

In general, an intersection over union metric specifies a percentage of correctly predicted edges, faces, and vertices within the predicted mesh, after an operation, such as completion of segmentation.

An average boundary distance specifies a distance between the predicted outputs 812 (or predicted representations 818) and the ground truth inputs 804 (or ground truth representations 820) for the first 3D representation 108 (such as the 3D mesh, the 3D point cloud, the voxelized representation, or the 3D surface).

A boundary percentage specifies the percentage of a mesh boundary length of the 3D mesh, such as a segmented 3D mesh, where the distance between the ground truth inputs 804 (or the ground truth representations 820) and the predicted outputs 812 (or the predicted representations 818) is below a threshold. For instance, the threshold can determine whether one or more of the predicted outputs 812, such as a small line segment between each pair of boundary points, is close enough to the ground truth input 804.

Where technique of segmenting the 3D representations shown in FIG. 8 is used to train the segmentation process, if the distance is below the threshold, then the line segment (e.g., or any other mesh element) may be labelled as a perfect boundary segment. The percentage represents a ratio of segments which reside within a boundary of the predicted output 812 compared to a boundary of the ground truth input 804. An over-segmentation ratio specifies the percentage of the mesh boundary length that the wound (or other facet of the skin 10 or the article 16 attached to the skin 10) is over-segmented, according to particular implementations. The one or more intersection over union metrics can be used to additionally train the generator 810 and/or the discriminator 822.

FIG. 9 is a flowchart of a method 900 for detecting an anomaly 1002 shown in FIG. 10, according to an implementation of the present disclosure. FIG. 10 is a schematic block diagram of a process 1000 of the method 900 shown in FIG. 9, according to an implementation of the present disclosure.

Referring to FIGS. 9 and 10, at step 902, the method 900 includes receiving the first 3D representation 108 that is representative of the clinical data 101.

At step 904, the method 900 includes providing the first 3D representation 108 as the input to the first ML module 304.

As discussed above, in some implementations, the first 3D representation 108 includes the one or more mesh elements 803 shown in FIG. 8, and the one or more mesh element features 303 are computed for at least one of the one or more mesh elements 803. In some implementations, the one or more mesh element features 303 are further provided to the first ML module 304.

At step 906, the method 900 includes executing the first ML module 304 to encode the first 3D representation 108 into the one or more latent representations 305 and reconstruct the one or more latent representations 305 into the second 3D representation 406 that is the facsimile of the first 3D representation 108.

At step 908, the method 900 includes computing the reconstruction error that quantifies a difference between the first 3D representation 108 and the second 3D representation 406.

At step 910, the method 900 includes determining at least one region 1004 of the first 3D representation 108 that has the reconstruction error greater than a predetermined threshold.

At step 912, the method 900 includes determining that the at least one region 1004 corresponds to the anomaly 1002. In some implementations, the anomaly 1002 includes at least one of the skin abnormality 20 (shown in FIG. 11A) and the article 16 (shown in FIG. 16) disposed on the skin 10 of the patient. In some implementations, the anomaly 1002 includes an anomalous material. The anomalous material may include an excess, dead, or scab material.

In some implementations, the method 900 may be implemented by the computing device 100 shown in FIG. 1 and may be integrated with a software application (e.g., a mobile application) and used in a treatment of the patient.

The first ML module 304 (e.g., a 3D mesh reconstruction variational autoencoder (VAE) with optional continuous normalizing flows) may be trained to identify the anomaly 1002 (e.g., an anomalous material) in the first 3D representation 108 (e.g., to identify damaged, infected, or wounded tissue in the first 3D representation 108 of the clinical data 101). For example, the 3D mesh reconstruction VAE may be trained to reconstruct examples of the first 3D representations 108 that are expected, e.g., healthy clinical data, such as one or more of healthy skin, healthy tissue, healthy appendage, or the like.

After training, when the first 3D representation 108 which is unexpected is presented to the 3D mesh reconstruction VAE, the reconstructed mesh (i.e., of the second 3D representation 406) may have a high reconstruction error. In some instances, the high reconstruction error may be localized to the mesh elements 803 which are associated with an anomalous portion (i.e., the at least one region 1004) of the unexpected first 3D representation 108 (e.g., the high reconstruction error may flag the mesh elements 803 which are associated with damage to the skin 10 or have an anomalous skin growth) under conditions where the 3D mesh reconstruction VAE was trained entirely on examples of the healthy (or otherwise non-anomalous) clinical data.

Once the anomaly 1002 is identified, the treatment can be rendered by a clinician. In some instances, in the case of the anomalous material which may be present in the vicinity of the wound or any other break in the skin 10, the anomalous material may be targeted for debridement.

Using the 3D mesh reconstruction VAE to identify the anomaly 1002 may facilitate determination of an excision margin and/or a depth (e.g., in the case the anomaly 1002 is a skin growth that is to be removed). In some instances, an offset boundary may be computed around a subset of the mesh elements 803 which are identified as anomalous (i.e., the anomaly 1002). Such a boundary may be used as the excision margin.

The first ML module 304 is trained to encode the first 3D representation 108 (or a list of mesh elements which correspond to the first 3D representation 108) using the encoder 402, yielding the one or more latent representations 305 (e.g., the one or more latent vectors). The one or more latent representations 305 may then be reconstructed into the second 3D representation 406 (i.e., the facsimile of the received first 3D representation 108). This reconstructed second 3D representation 406 may then be compared to the input 3D representation (i.e., the first 3D representation 108) using a reconstruction loss calculation, a KL-Divergence loss calculation, or other losses described herein to compute the reconstruction error. One or more loss values (i.e., the reconstruction error) may be used to train, at least in part, the encoder 402 or the decoder 404.

Through the course of training, this 3D mesh reconstruction VAE gets very good at reconstructing (e.g., with low reconstruction error) the 3D meshes of the first 3D representation 108 which reflect the distribution of the training dataset (e.g., the healthy clinical data).

When the first 3D representation 108 is introduced which contains an anomalous geometry and/or an anomalous structure, then the 3D mesh reconstruction VAE may struggle to deconstruct and reconstruct that first 3D representation 108. As a result, the reconstruction error may be a high value or greater than the predetermined threshold, which may flag a presence of the anomaly 1002.

Similarly, the reconstruction error (e.g., a 2D reconstruction error may be determined by comparing an input 2D image (i.e., the first 2D representation) and its corresponding reconstructed image.

In some implementations, the one or more mesh element features 303 (e.g., mesh dimension information) may be provided to the encoder 402, to improve an accuracy of the one or more latent representations 305. Similarly, the image texture features may be provided to the encoder 402 to improve the accuracy of the one or more latent representations 305. In some implementations, the first 2D representation of thermochromic dye on the skin 10 may reveal temperature.

FIG. 11A shows different types of the skin abnormalities 20 that can be classified by the second ML module 306 (shown in FIG. 3). In the illustrated examples of FIG. 11A, the skin abnormalities 20 are different types of the wounds.

Referring to FIGS. 3, 5, and 11A, as discussed above, the first 2D representation or the first 3D representation 108 of the clinical data 101 including the skin abnormality 20 (e.g., burn, incision, sore, wound, puncture, bruise, other skin injury, rash, infection, or cyst) may be provided to the first ML module 304, which may generate the corresponding one or more latent representations 305, which may be provided to the second ML module 306, which may classify the one or more latent representations 305 of the skin abnormality 20 according to the type of the skin abnormality 20. Furthermore, the second ML module 306 may classify the skin abnormality 20 according to a degree of clinical concern or severity of the skin abnormality 20: such as ‘superficial’ (e.g., shown in extreme left image of FIG. 11A), ‘moderate’ (shown in middle image of FIG. 11A), or ‘serious’ (shown in extreme right image of FIG. 11A). In some implementations, the degree of clinical concern or severity may be classified by the second ML module 306, for example, on a scale having 5 or 10 levels.

In some implementations, the skin abnormality 20 may be an infection. Thus, the second ML module 306 may classify the infection according to clinical categories, such as infected, not infected, or gangrenous.

Further, in some cases, the skin abnormality 20 may be an opening in the skin 10. The first 2D representation or the first 3D representation 108 of the skin 10 which includes the opening in the skin 10 may be provided to the first ML module 304. Further, the second ML module 306 may classify the corresponding one or more latent representations 305 according to clinical categories, such as opening due to a surgical incision, a burn, a gunshot, a puncture wound from various kinds of objects, and so forth. Such a classifier could be used in an emergency room, a battlefield, a fire scene, etc.

In some implementations, the first 3D representation 108 including the 3D mesh of the skin 10 of the patient may be generated, and subsequently provided to the first ML module 304 which includes, for example, the encoder 402 (e.g., such as may be trained as a part of the 3D VAE with optional continuous normalizing flows), which may generate the one or more latent representations 305 of the 3D mesh. The second ML module 306 may classify the one or more latent representations 305 of the skin abnormality 20, to identify the type of the skin abnormality 20. The 3D analysis of the skin abnormality 20 may entail information of the skin abnormality 20, such as a precise shape, a depth, or a texture of the skin abnormality 20, which may assist the second ML module 306 in the classification determination.

Further, when the first 3D representation 108 of the clinical data 101 including the skin abnormality 20 is analyzed, in some implementations, the one or more mesh element features 303 may be computed for the one or more mesh elements 803 of the first 3D representation 108. The one or more mesh element features 303 may, in some implementations, be provided to the first ML module 304, to improve an accuracy of the one or more latent representations 305 generated by the first ML module 304.

Further, as discussed above, in some implementations, at least one of the one or more mesh elements 803 has the at least one associated meta data value including the data pertaining to at least one of the color of the object, the temperature of the object, and the surface impedance of the object, or some other measured value (e.g., a value associated with the object which has been measured by a sensor). For example, a color (e.g., expressed as HSV or RGB) may be associated with the one or more mesh elements 803 of the first 3D representation 108. In some implementations, the associated meta data value including the data pertaining to the color may be provided to the first ML module 304 to improve the accuracy of the one or more latent representations 305. The object may, in some implementations, be illuminated by either UV or IR light. The color information, which is derived from the resulting images may, in some implementations, be associated with the one or more mesh elements 803 as the associated meta data value. In some implementations, data from an X-ray, a CT scan, an MRI scan, a fMRI scan, or other type of medical scan may be associated with the one or more mesh elements 803 as the associated meta data value.

In some implementations, the at least one associated meta data value including the data pertaining to at least one of the color of the object, the temperature of the object, and the surface impedance of the object may also be provided to the second ML module 306 to aid in classification and/or improve the classification accuracy.

For example, the data pertaining to the color (e.g., color pixels in the first 2D representation, or color as the mesh element feature 303 in the first 3D representation 108) may aid the classification of the burns, the bruises, the skin rashes, or the skin infections, among other of the skin abnormalities 20. Further, the data pertaining to the temperature may aid to classify poor blood flow in extremities or to classify vascular access sites as ‘normal/healthy’, ‘infected’, ‘phlebitis’, etc.

In some implementations, the second ML module 306 may classify the status or the current state of the skin abnormality 20, such as the broken or the injured skin (i.e., from the wound, the puncture, the incision, the burn, the bruises, or the like) at an individual time point based on the first 3D representation 108 of the broken or the injured skin. In some implementations, the second ML module 306 may be trained (e.g., according to techniques described herein) to label the one or more mesh elements 803 to identify a healthy tissue, for example, a patch of healthy skin adjacent to the broken or the injured skin.

Referring to FIGS. 5 and 11A, the second ML module 306 may be trained on a dataset where at least one example of the clinical data 101 reflects a state where the skin 10 has an abnormality (i.e., the skin abnormality 20, an attached article or a foreign object, etc.), and at least one example of the clinical data 101 reflects a state where the skin 10 is abnormality-free. The second ML module 306 may be trained, at least in part, by computing a loss (e.g., the loss 504 shown in FIG. 5) which compares a predicted class label (e.g., the at least one predicted classification label 308) to a reference class label (or the at least one ground truth classification label 502). Losses such as cross-entropy or others described herein may be computed as a part of the training.

The first ML module 304 may be used to segment the first 3D representation 108 of the skin 10 with the opening in the skin 10. This segmentation may classify the mesh elements 803 as an ‘opening in the skin’, or ‘other kind of skin abnormality’, ‘healthy skin surrounding the opening’, or any other category. Upon completion of this labeling, the excess material may be removed using mesh processing techniques. The resulting cleaned-up mesh may be classified according to the process 300 shown in FIG. 3. In some implementations, categories of the openings may include a healing incision, an incision dehiscence, an abscess drain, a catheter.

FIG. 11B shows identification of different locations of the skin abnormalities 20, according to an implementation of the present disclosure. Specifically, FIG. 11B shows identification of the different locations of the skin abnormalities 20 that are different types of the wounds.

Referring to FIGS. 8, 9, 10, and 11B, segmentation techniques described for the segmentation process 800 or the process 1000 may be applied to the skin abnormalities 20 described herein, for example, to quantify a size of the skin abnormality 20, or to show a progress in healing of the skin abnormality 20 over time. The first 3D representation 108 of the skin 10 of the patient (e.g., a portion of the skin 10 including the skin abnormality 20—such as the rash, the infection, the cut or the incision, the bruise, or the like) may be segmented to isolate an area of the abnormal portion of the skin 10 including the skin abnormality 20. The area of the skin abnormality 20 may be quantified at one or more time points, using the segmentation techniques described herein. In some implementations, the areas may be plotted over time to show the progress in healing of the skin abnormality 20 (as shown in FIG. 11C). The areas may be surrounded in a bounding box 1110.

FIG. 11C shows different stages of the skin abnormalities 20 that can be classified by the second ML module 306 (shown in FIG. 3), according to an implementation of the present disclosure. Specifically, in FIG. 11C, the skin abnormality 20 is the wound. As depicted in FIG. 11C (from left to right)), the wound is healing over time, for example, due to the treatment (e.g., a dressing, a topical antibiotic, an oral antibiotic, stem cells, endothelial cells, fibroblast growth factors, steroids, or hepatocyte growth factors, and so forth).

Referring to FIGS. 3, 4, and 11C, the second ML module 306 may, in some implementations, be trained to assess the state of the skin abnormality 20 on the skin 10 (or the appendage 12), and/or detect the skin abnormalities 20. The second ML module 306 may, in some implementations, analyze the skin 10 (or the appendage 12) over multiple time points. In some implementations, a state of recovery may be assessed, for example, after an application of the treatment based on one or more of the first 2D representations or the first 3D representations 108 of the clinical data 101 including the broken, incised, burned, rashed, bruised, infected, or otherwise injured skin.

In some instances, a healing of tissue after a medical procedure (such as surgery) may be assessed over one or more time points using the described herein. For example, the first 3D representation 108 of a healing incision (e.g., with optional data pertaining to the color) may be provided to the first ML module 304, which may encode the first 3D representation 108 into the one or more latent representations 305 (i.e., the latent vector form) using the encoder 402. The one or more latent representations 305 may be provided to the second ML module 306 which has been trained to classify the incisions into classes such as “fresh incision”, “initial healing underway”, or “fully healed”.

In an example, the healing of the skin abnormality 20, such as the wound, may be monitored. In some implementations, the progress in the healing may be expressed in terms of surface area, or a percentage area of a body part including the skin abnormality 20.

Referring to FIGS. 3, 5, and 11C, the second ML module 306 may be trained to predict the future state of the skin abnormality 20, based on a tuple including either the first 2D representation or the first 3D representation 108 from one or more time points. For example, a training dataset may include past clinical data (such as the clinical data 101) including many tuples.

Each tuple may include one or more 2D images or one or more of the first 3D representations 108 (optionally with the one or more mesh elements 803 having the at least one associated meta data value) of the skin 10 of the patient, where the skin abnormality 20 is included in at least one of the first 3D representations 108.

Each tuple may include a ground truth label information (e.g., the ground truth classification label 502), providing an indication of the future state (or healing trajectory) of the skin abnormality 20 depicted by the one or more of the first 3D representations 108 (e.g., ‘healing’ or ‘improving’, ‘not changing’, ‘getting worse’, and so forth).

The first ML module 304 may be trained to generate the one or more latent representations 305 from each of the first 3D representations 108 in the tuple (e.g., to generate the latent representation 305 of the 3D mesh of the incision or the wound with the one or more mesh element features 303 that are associated with the one or more mesh elements 803 optionally having has the at least one associated meta data value).

In some implementations, the one or more latent representations 305 associated with the tuple may be provided to the second ML module 306, which may be trained to generate the at least one predicted classification label 308, such as ‘healing’ or ‘improving’, ‘not changing’, ‘getting worse’, and so forth. The latent representations 305 for multiple time points may be provided to the second ML module 306 which includes a transformer.

The transformer may be provisioned to consume multiple inputs (i.e., the latent representations 305), and may be trained to generate a determination regarding whether a condition of the patient over two or more time points indicates that the skin 10 of the patient is ‘healing’ or ‘improving’, ‘not changing’, or ‘getting worse’.

FIG. 12A shows different types of the skin abnormalities 20 that can be classified by the second ML module 306 (shown in FIG. 3), according to another implementation of the present disclosure. In the illustrated examples of FIG. 12A, the skin abnormalities 20 are different types of the skin growths, such as the tumors.

A partial list of categories of the skin growths includes: skin tags (acrochordons), warts, dermatofibromas, dermoid cyst, birthmarks (such as hemangiomas, port-wine stains), freckles, keloids, keratoacanthomas, lipomas, moles (nevi), atypical moles (dysplastic nevi), seborrheic keratoses, melanoma, basal cell carcinoma, squamous cell carcinoma, and cutaneous horns.

Referring to FIGS. 3, 5, and 12A, in some implementations, the second ML module 306 may be trained to classify a state of one or more tumors at an individual time point based on the first 3D representation 108 of the clinical data 101 including the one or more tumors.

In some implementations, the second ML module 306 may be used to label a portion of the skin 10, such as the skin 10 including the one or more tumors, according to a state of health of that portion of the skin 10.

In some instances, the second ML module 306 may be trained on 3D representations to apply a label of ‘cancerous’ or ‘benign’ to a 3D representation (i.e., the first 3D representation 108) of the tumor. In some instances, the second ML module 306 may be trained on 3D representations to apply a label of ‘dangerous’ or ‘not dangerous’ to the 3D representation of the tumor.

The second ML module 306 may be trained to classify the first 3D representation 108 of the skin 10 (or the appendage 12) including the tumors, and/or healthy tissue next to the tumors, and predict the at least one predicted classification label 308 for the first 3D representation 108. The at least one predicted classification label 308 may include but are not limited to: ‘cancerous’ or ‘benign’. In some implementations, an ML model for mesh element labeling may be trained to label mesh elements as either ‘cancerous’ or ‘benign’, for example, by training the ML model as shown and described with respect to FIG. 4.

FIG. 12B shows identification of different locations of the skin abnormalities 20, according to another implementation of the present disclosure. Specifically, FIG. 12B shows identification of the different locations of the skin abnormalities 20 that are different types of the skin growths.

As shown in FIG. 12B, in some implementations, the skin growths with a cancerous growth may be surrounded in a bounding box 1210 and another growth that is not identified as the cancerous growth may not be surrounded in the bounding box 1210. Alternatively, in some implementations, the skin growths with the cancerous growth are surrounded in the bounding box 1210 and a benign/or the non-cancerous growth is surrounded by a circle 1220. In some other implementations, different types of the tumors or the skin growth may be surrounded by any enclosed shape, such as a rectangle, a square, an oval, a polygon, as per desired application attributes.

FIG. 12C shows different stages of the skin abnormality 20 that can be classified by the second ML module 306 (shown in FIG. 3), according to another implementation of the present disclosure. Specifically, in FIG. 12C, the skin abnormality 20 is the skin growth. As depicted in FIG. 12C, the skin growth is shrinking over time, for example, due to the treatment (e.g., such as a radiation, a chemotherapy, or after surgical removal).

Similarly, as discussed with reference to FIG. 11C, in a further example, a status of the tumor may be predicted using the tuple including data from multiple time points (taken over the course of days, weeks, months, etc.). In some implementations, a state of change of the one or more tumors may be assessed, for example, after an application of the treatment. The second ML module 306 may be trained using the process 500 shown in FIG. 5 to classify one or more of the first 3D representations 108 including the tumors, and/or the healthy tissue next to the tumors, and predict one or more class labels (the at least one predicted classification label 308) for the one or more of the first 3D representation 108. The at least one predicted classification label 308 may include but are not limited to: ‘tumor is getting larger’, ‘tumor is getting smaller’, or ‘tumor is not changing in size’.

FIG. 13 shows the implant site 13 including the implant 14 on the skin 10, according to an implementation of the present disclosure. In the illustrated implementation of FIG. 13, the implant 14 is the device implant.

Referring to FIGS. 3 and 13, in some implementations, the second ML module 306 may classify a state of the implant site 13 on the skin 10, based on one or more of the first 3D representations 108 of the skin 10 and/or the implant 14. Such clinical data analysis may be performed at one time point or over a series of time points, for example, to monitor a healing process after the implantation of the implant 14.

The first 3D representation 108 of the implant site 13 on the skin 10 and/or the implant 14 may be provided to the first ML module 304, which may generate the one or more latent representations 305 of the first 3D representation 108. The one or more latent representations 305 may be provided to the second ML module 306, which may be trained to classify the one or more latent representations 305. The second ML module 306 may generate the at least one predicted classification label 308 pertaining to the implant site 13, including (but not limited to): infected, healthy, accepted, rejected, healing, not healing, dehiscence, or the like.

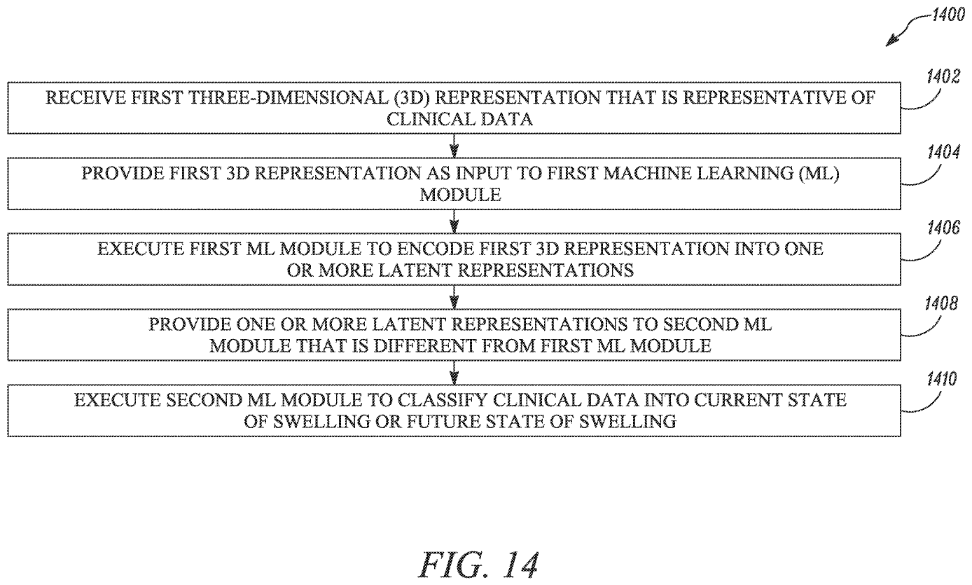

FIG. 14 is a flowchart of a method 1400 for detecting the swelling 22 shown in FIG. 15, according to an implementation of the present disclosure. FIG. 15 shows different states/stages of the swelling 22 in the appendage 12, according to an implementation of the present disclosure. In some implementations, the method 1400 is used to detect lymphedema.

Referring to FIGS. 3, 5, 14, and 15, at step 1402, the method 1400 includes receiving the first 3D representation 108 that is representative of the clinical data 101. The clinical data 101 is representative of the skin 10 of the patient or the appendage 12 of the patient.

At step 1404, the method 1400 includes providing the first 3D representation 108 as the input to the first ML module 304.

At step 1406, the method 1400 includes executing the first ML module 304 to encode the first 3D representation 108 into the one or more latent representations 305.

At step 1408, the method 1400 includes providing the one or more latent representations 305 to the second ML module 306 that is different from the first ML module 304.

At step 1410, the method 1400 includes executing the second ML module 306 to classify the clinical data 101 into the current state of the swelling 22 or the future state of the swelling 22. In some implementations, the current state of the swelling 22 or the future state of the swelling 22 is used to detect lymphedema.

In some implementations, receiving the first 3D representation 108 includes receiving at least two first 3D representations that are representative of the clinical data 101 obtained at different time intervals.

In some implementations, providing the first 3D representation 108 as the input to the first ML module 304 includes providing the at least two first 3D representations as the input to the first ML module 304.

In some implementations, executing the first ML module 304 to encode the first 3D representation 108 into the one or more latent representations 305 includes executing the first ML module 304 to encode the at least two first 3D representations into corresponding one or more latent representations.

In some implementations, providing the one or more latent representations 305 to the second ML module 306 includes providing the corresponding one or more latent representations to the second ML module 306.

In some implementations, executing the second ML module 306 to classify the clinical data 101 into the current state of the swelling 22 or the future state of the swelling 22 includes comparing the corresponding one or more latent representations by the second ML module 306 to classify the clinical data 101 into the current state of the swelling or the future state of the swelling.

The first 2D representation or the first 3D representation 108 of the appendage 12 (e.g., the finger, the arm, the leg, the foot, the hand, or the like) or the torso (e.g., the groin, the abdomen, the chest, the shoulders, or the like) of the patient may be provided to the first ML module 304, which may generate the corresponding one or more latent representations 305, which may be provided to the second ML module 306, which may classify the corresponding one or more latent representations 305 according to clinical categories, such as ‘swelling is present’, or ‘swelling is not present’. In some implementations, a degree of clinical concern or severity of the swelling 22 may be classified by the second ML module 306, for example, on a scale having 5 or 10 levels.

In some implementations, the method 1400 may be used to inform the clinician on a severity of the swelling 22. In some instances, the clinical data 101 pertaining to the appendage or the torso of the patient may be collected at multiple time points.

The second ML module 306 may be trained on a dataset where at least one example of the clinical data 101 reflects that the swelling 22 is not present, and at least one example of the clinical data 101 reflects a state where the swelling 22 is present.

The second ML module 306 may be trained, at least in part, by computing a loss (e.g., the loss 504) which compares a predicted class label (e.g., the at least one predicted classification label 308) to a reference class label (e.g., the at least one ground truth classification label 502). The losses such as the cross-entropy or the others described herein may be computed as a part of the training.

In some instances, the appendage 12 or the torso may undergo scanning (e.g., scanning to generate the first 3D representation 108) when the appendage 12 or the torso does not include the swelling 22. At subsequent time points, the appendage 12 or the torso of the patient may be rescanned. The first 3D representation 108 from a given time point may be provided to the first ML module 304, which may then generate the one or more latent representations 305.

The one or more latent representations 305 may then be provided to the second ML module 306, which may classify the first 3D representation 108 according to the current state of the swelling 22 at that time point. This clinical data analysis of the appendage 12 or the torso over multiple time points may be applied to a treatment of lymphedema, which affects the lymph system of the patient and causes fluid to accumulate in a soft tissue of the appendage 12 or the torso. The patient may use the method 1400 to detect the swelling 22 over the course of a day. When the swelling 22 is detected, the patient may apply a wrap or a compression garment.

FIG. 16 shows the article 16 disposed on the skin 10 of the patient, according to an implementation of the present disclosure. Specifically, in the illustrated implementation of FIG. 16, the article 16 is the wrapping 18 on the skin 10 of the patient.

FIG. 16 depicts different fits of the wrapping 18 on the skin 10 of the patient.

Specifically, FIG. 16 depicts the wrappings 18 having different tensions on the skin 10 of the patient. The fit of the wrapping 18 on the skin 10 of the patient may be a loose fit/tension (shown in leftmost image of FIG. 16), a correct fit/tension (shown in middle image of FIG. 16), or a tight fit/tension (shown in rightmost image of FIG. 16). It may be noted that a texture of the wrapping 18 may change with the change in the fit/tension of the wrapping 18 on the skin 10 of the patient. Therefore, the texture of the wrapping 18 may be helpful in assessing a proper fit/tension of the wrapping 18 on the skin 10.

The first 2D representation (e.g., one or more images) or the first 3D representation 108 of the appendage 12 or any other body part of the patient with the wrapping 18 attached on the skin 10 may be provided to the first ML module 304, which may generate the corresponding one or more latent representations 305, which may be provided to the second ML module 306, which may classify the corresponding one or more latent representations 305 of the appendage 12 or the other body part with the wrapping 18 according to a current state of the wrapping 18 in clinical categories, such as ‘too tight’, ‘tight’, ‘loose’, ‘too loose’, or ‘just right’, and so forth.

In some implementations, the segmentation techniques described for the segmentation process 800 or the process 1000 may be performed on the first 3D representation 108 (e.g., the 3D mesh) of the wrapping 18 attached on the skin 10 to label the mesh elements 803 as ‘healthy skin’, ‘wrapping’, or some other category. The portions of the 3D mesh belonging to the wrapping 18 may be fed into the first ML module 304, so that mesh classification may be performed.

As discussed above, in some implementations, the wrapping 18 may be the dressing. In such implementations, the second ML module 306 may classify the one or more latent representations 305 of the dressing on the skin 10 of the appendage 12 or the other body part of the patient according to a current state of the dressing or the bandage in clinical categories, such as wrinkles are present, wrinkles are present which may lead to leaks or the introduction of infection, or wrinkles are not present. Wrinkles in the dressing may be predictive of leaks. Specifically, the wrinkles may lead to leaks. Therefore, techniques of this disclosure may be trained to analyze the wrinkles in the dressing or the bandage.

In some implementations, the segmentation techniques described for the segmentation process 800 or the process 1000 may be performed on the first 3D representation 108 (e.g., the 3D mesh) of the dressing and the skin 10 surrounding the dressing, to label the mesh elements 803 according to membership in respective portions of the 3D mesh (e.g., the healthy skin, the wound, dressing, etc.).

This kind of a segmentation process may further aid the clinical data analysis (e.g., the analysis which seeks to detect and classify the wrinkles which may lead to the leaks) by isolating portions of the 3D mesh which represent the dressing (or by isolating the specific portions of the 3D mesh which correspond to the wrinkles). Once the portions of the 3D mesh which represent the dressing are isolated, the process of identifying the wrinkles and predicting whether those wrinkles may lead to leaks may be greatly improved and strengthened (since a neural network for wrinkle classification can isolate and study the dressing).

In some instances, the segmentation process may further isolate one or more specific wrinkles on the dressing (e.g., apply a distinct label to each of the mesh element 803 which is found on a particular wrinkle, and apply a different label to each of the mesh element 803 which is found on a different wrinkle). This refined segmentation may further aid the neural network, which is trained to classify the wrinkles, as ‘likely to cause a leak’ or ‘unlikely to cause a leak’ (among other categories).

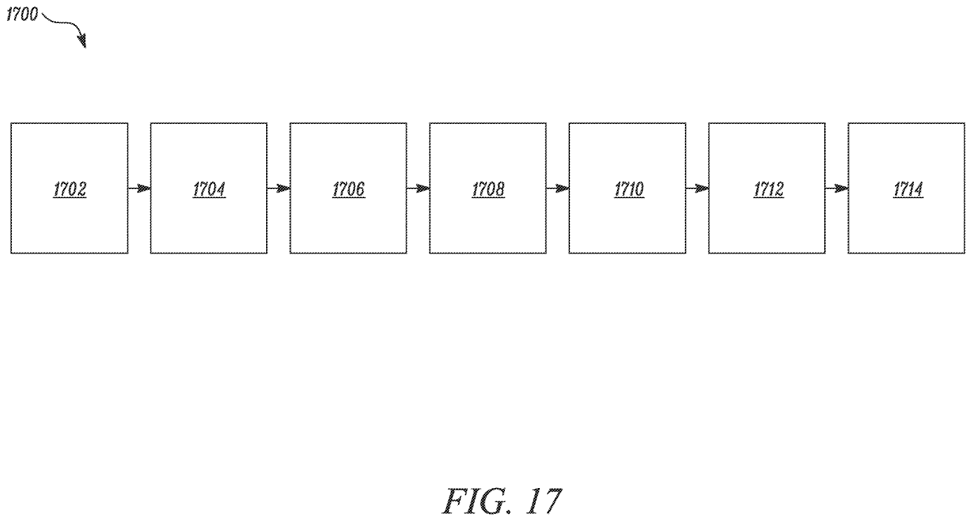

FIG. 17 is a schematic block diagram of a data augmentation process 1700 that may be applied to the one or more mesh elements 803 of the first 3D representation 108 of the clinical data 101 shown in FIG. 2, according to an implementation of the present disclosure.

Various neural network models of this disclosure may draw benefits from data augmentation. Data augmentation, such as by way of the data augmentation process 1700 shown in FIG. 17, may increase the size of a training dataset of a clinical data (e.g., the clinical data 101). Data augmentation can provide additional training examples by adding random rotations, translations, and/or rescaling to copies of the existing clinical data. In some implementations of the techniques of this disclosure, the data augmentation may be carried out by perturbing or jittering the vertices of the 3D mesh, in a manner similar to that described in (“Equidistant and Uniform Data Augmentation for 3D Objects”, IEEE Access, Digital Object Identifier 10.1109/ACCESS.2021.3138162). The position of a vertex may be perturbed through the addition of Gaussian noise, for example, with zero mean, and 0.1 standard deviation. Other mean and standard deviation values are possible in accordance with the techniques of this disclosure.

The data augmentation process 1700 may be used by systems (e.g., the computing device 100 shown in FIG. 1) to apply to the clinical data 101 shown in FIG. 2. As discussed above, a non-limiting example of the clinical data is the 3D mesh which describes contours of the wound or the skin 10 adjacent to the wound. At block 1702, the clinical data 101 (e.g., the 3D meshes) is received as an input. At block 1704, the systems of this disclosure may generate copies of the clinical data 101. At block 1706, the systems of this disclosure may apply one or more stochastic rotations to the clinical data 101. At block 1708, the systems of this disclosure may apply stochastic translations to the clinical data 101. At block 1710, the systems of this disclosure may apply stochastic scaling operations to the clinical data 101. At block 1712, the systems of this disclosure may apply stochastic perturbations to the one or more mesh elements 803 of the clinical data 101. At block 1714, the systems of this disclosure may output an augmented 3D clinical data that may be formed by way of the data augmentation process 1700 of FIG. 17.

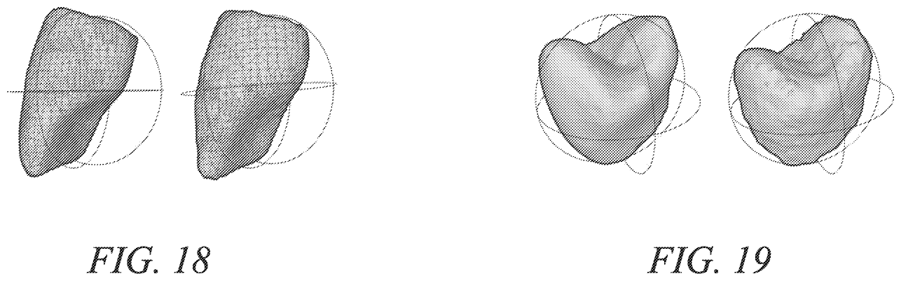

FIGS. 18 and 19 each shows an input 3D mesh on the left and corresponding reconstructed mesh on the right, according to an implementation of the present disclosure. Specifically, FIGS. 18 and 19 each shows the input 3D mesh of a tooth on the left and the corresponding reconstructed mesh of the tooth on the right. The first ML module 304 (i.e., the reconstruction autoencoder shown in FIG. 4) may be trained to reconstruct the first 3D representations 108 of the clinical data 101, such as the skin 10, the appendages 12, the wounds, the dressings, or types of anatomy (e.g., the tooth).

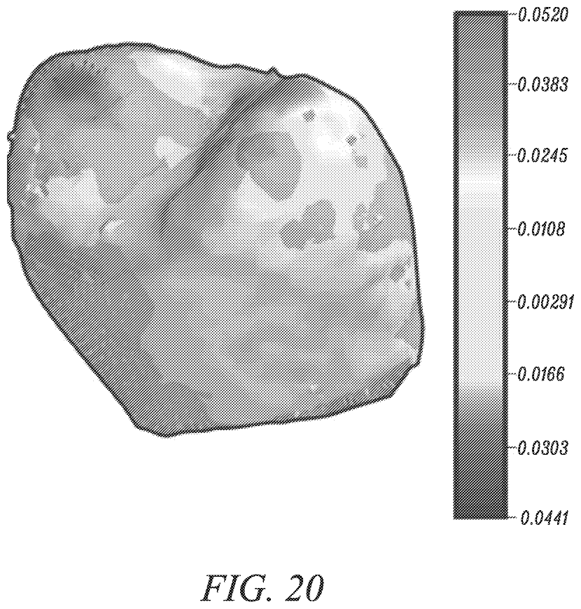

FIG. 20 shows a depiction of the reconstruction error from the reconstructed tooth, called a reconstruction error plot, according to an implementation of the present disclosure.

Specifically, FIG. 20 depicts the reconstruction error in the results described above with respect to FIGS. 18 and 19, in a form referred to as the reconstruction error plot with units in millimeters (mm). It is to be noted that the reconstruction error is less than 50 microns at cusp tips, and much less than 50 microns over most of the tooth surface. As compared to a typical tooth with a size of 1.0 cm, an error rate of 50 microns (or less) may mean that the tooth surface was reconstructed with an error rate of less than 0.5%.

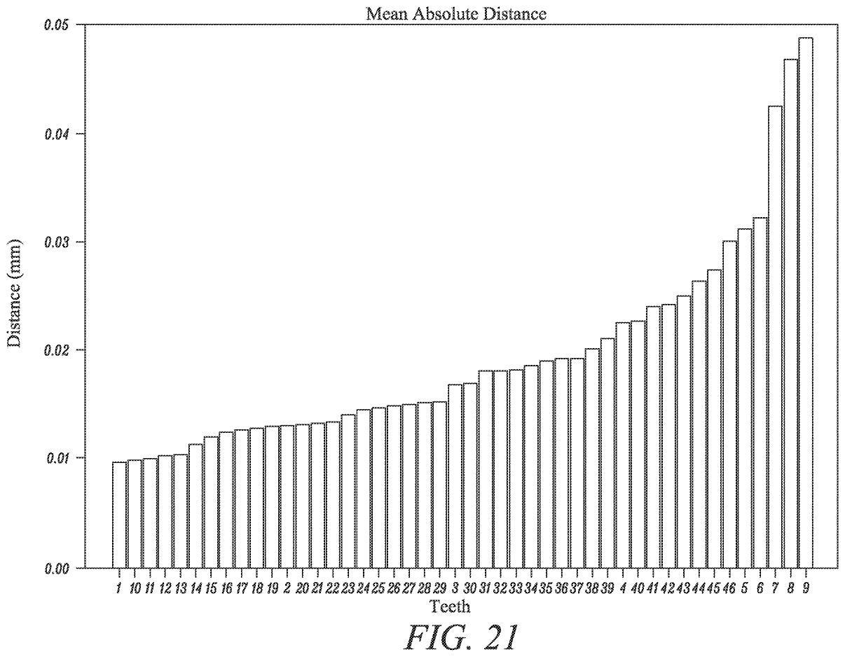

FIG. 21 is a bar chart in which each bar represents an individual tooth and represents mean absolute distance of all vertices involved in the reconstruction of that tooth in a data that was used to evaluate the performance of a reconstruction model (e.g., the first ML module 304).

Various loss calculation techniques are generally applicable to the techniques of this disclosure, for example, for calculating the reconstruction loss 408 (shown in FIG. 4) or the loss 504 (shown in FIG. 5).

These losses include the L1 loss and the L2 loss (as described above), mean squared error (MSE) loss, cross entropy loss, among others. The losses may be computed and used in the training of neural networks, such as multi-layer perceptron's (MLP), U-Net structures, generators, and discriminators (e.g., for GANs), autoencoders, variational autoencoders, regularized autoencoders, masked autoencoders, transformer structures, or the like. Some implementations may use either triplet loss or contrastive loss, for example, in the learning of sequences.

Losses may also be used to train encoder structures and decoder structures. A KL-Divergence loss may be used, at least in part, to train one or more of the neural networks of the present disclosure, which the advantage of imparting Gaussian behavior to the optimization space. This Gaussian behavior may enable a reconstruction autoencoder to produce a better reconstruction (e.g., when a latent vector representation is modified and that modified latent vector is reconstructed using a decoder, the resulting reconstruction is more likely to be a valid instance of the inputted representation). There are other techniques for computing losses which may be described elsewhere in this disclosure. Such losses may be based on quantifying the difference between two or more 3D representations.

The MSE loss calculation may involve calculation of an average squared distance between two sets, vectors, or datasets. MSE may be generally minimized. MSE may be applicable to a regression problem, where a prediction generated by a neural network or any other ML model may be a real number. In some implementations, a neural network may be equipped with one or more linear activation units on the output to generate an MSE prediction. Mean absolute error (MAE) loss and mean absolute percentage error (MAPE) loss can also be used in accordance with the techniques of this disclosure.

Cross entropy may, in some implementations, be used to quantify the difference between two or more distributions. Cross entropy loss may, in some implementations, be used to train the neural networks of the present disclosure. Cross entropy loss may, in some implementations, involve comparing a predicted probability to a ground truth probability. Other names of cross entropy loss include “logarithmic loss,” “logistic loss,” and “log loss”. A small cross entropy loss may indicate a better (e.g., more accurate) model. Cross entropy loss may be logarithmic. Cross entropy loss may, in some implementations, be applied to binary classification problems. In some implementations, a neural network may be equipped with a sigmoid activation unit at the output to generate a probability prediction. In the case of multi-class classifications, cross entropy may also be used. In such a case, a neural network trained to make multi-class predictions may, in some implementations, be equipped with one or more softmax activation functions at the output (e.g., where there is one output node for class that is to be predicted). Other loss calculation techniques which may be applied in the training of the neural networks of this disclosure include one or more of: Huber loss, Hinge loss, Categorical hinge loss, cosine similarity, Poisson loss, Logcosh loss, or mean squared logarithmic error loss (MSLE). Other loss calculation methods are described herein and may be applied to the training of any of the neural networks described in the present disclosure.

One or more of the neural networks of the present disclosure may, in some implementations, be trained, at least in part by a loss which is based on at least one of: a Point-wise Mesh Euclidean Distance (PMD) and an Earth Mover's Distance (EMD). Some implementations may incorporate a Hausdorff Distance (HD) calculation into the loss calculation. Computing the Hausdorff distance between two or more 3D representations (such as 3D meshes) may provide one or more technical improvements, in that the HD not only accounts for the distances between two meshes, but also accounts for the way that those meshes are oriented, and the relationship between the mesh shapes in those orientations (or positions or poses). Hausdorff distance may improve the comparison of two or more 3D clinical data, such as two or more instances of a 3D clinical data which are in different poses.

The techniques of this disclosure may include operations such as 3D convolution, 3D pooling, 3D unconvolution and 3D unpooling. 3D convolution may aid segmentation processing, for example, in down sampling a 3D mesh. 3D un-convolution undoes 3D convolution, for example, in a U-Net. 3D pooling may aid the segmentation processing, for example, in summarized neural network feature maps. 3D un-pooling undoes 3D pooling, for example, in a U-Net. These operations may be implemented by way of one or more layers in the predictive or generative neural networks described herein. These operations may be applied directly on mesh elements, such as mesh edges or mesh faces. These operations provide for technical improvements over other approaches because the operations are invariant to mesh rotation, scale, and translation changes. In general, these operations depend on edge (or face) connectivity, therefore these operations remain invariant to mesh changes in 3D space as long as edge (or face) connectivity is preserved. That is, the operations may be applied to a 3D clinical data and produce the same output regardless of an orientation, a position, or a scale of that 3D clinical data, which may lead to data precision improvement. MeshCNN is a general-purpose deep neural network library for 3D triangular meshes, which can be used for tasks such as 3D shape classification or mesh element labelling (e.g., for segmentation or mesh cleanup). MeshCNN implements these operations on mesh edges. Other toolkits and implementations may operate on edges or faces.

In some implementations of the techniques of this disclosure, neural networks may be trained to operate on 2D representations (e.g., the first 2D representation including the one or more images). In some implementations of the techniques of this disclosure, the neural networks may be trained to operate on 3D representations (e.g., the first 3D representation 108 including the 3D meshes or the 3D point clouds). An imaging device may capture 2D images of aspects of the body of the patient from various views. A 3D scanner may also (or alternatively) capture the 3D mesh or the 3D point cloud which describes the aspects of the body of the patient. According to various techniques, the autoencoders (or other neural networks described herein) may be trained to operate on either or both of the 2D representations and the 3D representations.

The 2D autoencoder (comprising a 2D encoder and a 2D decoder) may be trained on 2D image data to convert an input 2D image into a latent form (such as a latent vector or a latent capsule) using the 2D encoder, and then reconstruct a facsimile of the input 2D image using the 2D decoder. In the case of a handheld mobile app which has been developed for such analysis (e.g., for analysis of the clinical data), 2D images may be readily captured using one or more of the onboard cameras. In other examples, the 2D images may be captured using a 2D scanner which is configured for such a function. Among the operations which may be used in the implementation of the 2D autoencoder (or any other 2D neural network) for 2D image analysis are 2D convolution, 2D pooling and 2D reconstruction error calculation.

The 2D convolution may involve a ‘sliding’ of a kernel across a 2D image and the calculation of elementwise multiplications and the summing of those elementwise multiplications into an output pixel. The output pixel that results from each new position of the kernel is saved into an output 2D feature matrix. In some implementations, neighboring elements (e.g., pixels) may be in well-defined locations (e.g., above, below, left, and right) in a rectilinear grid.

A 2D pooling layer may be used to down sample a feature map and summarize a presence of certain features in that feature map.

2D reconstruction error may be computed between the pixels of the input and reconstructed images. The mapping between the pixels may be well understood (e.g., the upper pixel [23,134] of the input image is directly compared to pixel [23,134] of the reconstructed image, assuming both images have the same dimensions).

Among the advantages provided by the 2D autoencoder-based techniques of this disclosure is the ease of capturing 2D image data with a handheld device. In some instances, where outside data sources provide the data for analysis, there may be instances where only 2D image data are available. When only 2D image data are available, then analysis using the 2D autoencoder is warranted.

Modern mobile devices (such as commercially available smartphones) may also have the capability of generating 3D data (e.g., using multiple cameras and stereophotogrammetry, or one camera which is moved around the subject to capture multiple images from different views, or both), which in some implementations, may be arranged into the 3D representations such as the 3D meshes, the 3D point clouds and/or the 3D voxelized representations. The analysis of the 3D representation of the subject may in some instances provide technical improvements over the 2D analysis of the same subject. For example, a 3D representation may describe a geometry and/or a structure of the subject with less ambiguity than a 2D representation (which may include shadows and other artifacts which complicate depiction of a depth from the subject and a texture of the subject). In some implementations, 3D processing may enable technical improvements because of an inverse optics problem which may, in some instances, negatively affect the 2D representations. The inverse optics problem refers to the phenomenon where, in some instances, the size of a subject, the orientation of the subject, and the distance between the subject and the imaging device may be conflated in a 2D image of that subject. Any given projection of the subject on an imaging sensor of the imaging device could map to an infinite count of {size, orientation, distance} pairings. The 3D representations may enable the technical improvement in that the 3D representations remove the ambiguities introduced by the inverse optics problem.

A device that is configured with the dedicated purpose of 3D scanning, such as a 3D scanner (or a CT scanner or MRI scanner), may generate the 3D representations of the subject (e.g., the aspects of the body of the patient) which have significantly higher fidelity and precision than is possible with the handheld device. When such high-fidelity 3D data are available (e.g., in the application of classification techniques described herein), the use of the 3D autoencoder offers technical improvements (such as increased data precision), to extract the best possible signal out of those 3D data.

The 3D autoencoder (comprising a 3D encoder and a 3D decoder) may be trained on the 3D data representations to convert an input 3D representation into a latent form (such as a latent vector or a latent capsule) using the 3D encoder, and then reconstruct a facsimile of the input 3D representation using the 3D decoder. Among the operations which may be used to implement the 3D autoencoder for the analysis of a 3D representation (e.g., the 3D mesh or the 3D point cloud) are 3D convolution, 3D pooling and 3D reconstruction error calculation.

For each mesh element, the 3D convolution may be performed to aggregate local features from nearby mesh elements. Processing may be performed above and beyond the techniques for 2D convolution, to account for the differing count and locations of neighboring mesh elements (relative to a particular mesh element). A particular 3D mesh element may have a variable count of neighbors and those neighbors may not be found in expected locations (as opposed to a pixel in 2D convolution which may have a fixed count of neighboring pixels which may be found in known or expected locations). In some instances, the order of neighboring mesh elements may be relevant to 3D convolution.

A 3D pooling operation may enable the combining of features from a 3D mesh (or other 3D representation) at multiple scales. The 3D pooling may iteratively reduce the 3D mesh into mesh elements which are most highly relevant to a given application (e.g., for which a neural network has been trained). Similar to the 3D convolution, the 3D pooling may benefit from special processing beyond that entailed in the 2D convolution, to account for the differing count and locations of neighboring mesh elements (relative to a particular mesh element). In some instances, the order of neighboring mesh elements may be less relevant to the 3D pooling than to the 3D convolution.

3D reconstruction error may be computed using one or more of the techniques described herein, such as computing Euclidean distances between corresponding mesh elements, between the two meshes. Other techniques are possible in accordance with aspects of this disclosure. The 3D reconstruction error may generally be computed on 3D mesh elements, rather than the 2D pixels of the 2D reconstruction error. The 3D reconstruction error may enable technical improvements over the 2D reconstruction error, because a 3D representation may, in some instances, have less ambiguity than a 2D representation (i.e., have less ambiguity in form, a shape and/or a structure). Additional processing may, in some implementations, be entailed for the 3D reconstruction which is above and beyond that of the 2D reconstruction, because of the complexity of mapping between the input and reconstructed mesh elements (i.e., the input and reconstructed meshes may have different mesh element counts, and there may be a less clear mapping between mesh elements than there is for the mapping between pixels in the 2D reconstruction). The technical improvements of the 3D reconstruction error calculation include data precision improvement.

A 3D representation may be produced using a 3D scanner, a computerized tomography (CT) scanner, an ultrasound scanner, a magnetic resonance imaging (MRI) machine, or a mobile device which is enabled to perform stereophotogrammetry. A 3D representation may describe a shape and/or a structure of a subject. The 3D representation may include one or more of a 3D mesh, a 3D point cloud, and/or a 3D voxelized representation, among others. The 3D mesh includes edges, vertices, or faces. Though interrelated in some instances, these three types of data are distinct. The vertices are points in a 3D space that define boundaries of the 3D mesh. These points would alternatively be described as a point cloud but for the additional information about how the points are connected to each other, as described by the edges. The edge is described by two points and can also be referred to as a line segment. The face is described by a number of edges and vertices. For instance, in the case of a triangle mesh, the face comprises three vertices, where the vertices are interconnected to form three contiguous edges. Some 3D meshes may include degenerate elements, such as non-manifold mesh elements, which may be removed, to benefit of later processing. Other mesh pre-processing operations are possible in accordance with aspects of this disclosure. The 3D meshes are commonly formed using triangles, but may in other implementations be formed using quadrilaterals, pentagons, or some other n-sided polygon. In some implementations, the 3D mesh may be converted to one or more voxelized geometries (i.e., comprising voxels), such as in the case that sparse processing is performed. The techniques of this disclosure which operate on the 3D meshes may receive as input one or more meshes describing 3D clinical data (e.g., a skin of a patient with an attached article). Each of these meshes may undergo pre-processing before being input to the predictive architecture (e.g., including at least one of an encoder, a decoder, a pyramid encoder-decoder, and a U-Net). This pre-processing may include conversion of the mesh into lists of mesh elements, such as the vertices, the edges, the faces or in the case of sparse processing—the voxels. For the chosen mesh element type or types, (e.g., the vertices), feature vectors may be generated. In some examples, one feature vector is generated per vertex of the mesh. Each feature vector may include a combination of spatial and/or structural features, as specified in the following table:

TABLE 1

| Element | Spatial Features | Structural Features |
|---|---|---|
| Edges | XYZ position of an edge | Edge curvature (depends on a |
| | midpoint, XYZ positions of | connectivity neighborhood, |
| | the edge vertices, or the | average curvature of two |
| | normal vector at an edge | vertices), dihedral angles, |
| | midpoint (average of the | edge length, density measure |
| | normal vectors of two | such as a count of incident |
| | vertices). | edges (i.e., a count of the |
| | | other neighboring edges |
| | | which share the vertices of |
| | | that edge). |
| Faces | XYZ position of a face | Face curvature (average |
| | centroid, surface normal | curvature of the vertices of |
| | vector. | the face), face area, density |
| | | measure such as count of |
| | | adjacent faces (i.e., which |
| | | share at least one edge with |
| | | the face). |
| Points | XYZ position | Density measure such as the |
| | | count of neighboring points |
| | | within a radius of the point |
| Vertices | XYZ position, normal vector | Vertex curvature, density |
| | (weighted average of the | measure such as the count of |
| | normal vectors of the | vertices within a radius of the |
| | connecting faces for the | vertex, density measure such |
| | vertex). | as the count of incident edges. |
| Voxels | XYZ centroid. | Volume, [height x depth x |
| | | width] dimensions, density |
| | | measure such as a count of |
| | | contained vertices, density |
| | | measure such as count of |
| | | intersected faces, density |
| | | measure such as count of |
| | | intersected edges. |

Table 1 discloses non-limiting examples of mesh element features. In some implementations, a color (or other visual cues/identifiers) may be considered as a mesh element feature in addition to the spatial or the structural mesh element features described in Table 1. The color may be expressed as RGB, HSV, or the like. As used herein (e.g., in Table 1), the point differs from the vertex in that the point is part of the 3D point cloud, whereas the vertex is part of the 3D mesh and may have incident faces or edges. A dihedral angle (which may be expressed in either radians or degrees) may be computed as an angle (e.g., a signed angle) between two connected faces (e.g., two faces which are connected along an edge). A sign on the dihedral angle may reveal information about a convexity or a concavity of a mesh surface. For example, a positively signed angle may, in some implementations, indicate a convex surface. Furthermore, a negatively signed angle may, in some implementations, indicate a concave surface. To calculate a principal curvature of a mesh vertex, directional curvatures may first be calculated to each adjacent vertex around the vertex. These directional curvatures may be sorted in circular order (e.g., 0, 49, 127, 210, 305 degrees) in proximity to a vertex normal vector and may comprise a subsampled version of a complete curvature tensor. Circular order means sorted in by angle around an axis. The sorted directional curvatures may contribute to a linear system of equations amenable to a closed form solution which may estimate the two principal curvatures and directions, which may characterize the complete curvature tensor.

Consistent with Table 1, a voxel may also have features which are computed as the aggregates of the other mesh elements (e.g., the vertices, the edges, and the faces) which either intersect the voxel or, in some implementations, are predominantly or fully contained within the voxel. Rotating the mesh may not change structural features but may change spatial features. And, as described elsewhere in this disclosure, the term “mesh” should be considered in a non-limiting sense to be inclusive of 3D mesh, 3D point cloud, and 3D voxelized representation. In some implementations, apart from mesh element features, there are alternative methods of describing the geometry of a mesh, such as 3D keypoints and 3D descriptors. Examples of such 3D keypoints and 3D descriptors are found in “TONIONI A, et al. in ‘Learning to detect good 3D keypoints.’, Int J Comput. Vis. 2018 Vol. 126, pages 1-20.”. The 3D keypoints and 3D descriptors may, in some implementations, describe extrema (either minima or maxima) of the surface of a 3D representation. In some implementations, the one or more mesh element features may be computed, at least in part, via deep feature synthesis (DFS), e.g., as described in: J. M. Kanter and K. Veeramachaneni, “Deep feature synthesis: Towards automating data science endeavors,” 2015 IEEE International Conference on Data Science and Advanced Analytics (DSAA), 2015, pp. 1-10, doi: 10.1109/DSAA.2015.7344858.

Representation generation neural networks based on autoencoders, U-Nets, transformers, other types of encoder-decoder structures, convolution & pooling layers, or other models may benefit from the use of mesh element features. The mesh element features may convey aspects of a 3D representation's surface shape and/or structure to the neural network models of this disclosure. Each mesh element feature describes distinct information about the 3D representation that may not be redundantly present in other input data that are provided to the neural network. For example, a vertex curvature may quantify aspects of the concavity or the convexity of the surface of the 3D representation which would not otherwise be understood by the network. Stated differently, the mesh element features may provide a processed version of the structure and/or the shape of the 3D representation, data that would not otherwise be available to the neural network. This processed information is often more accessible, or more amenable for the neural network to encode into the weights of the neural network. A system disclosing the techniques disclosed herein has been utilized to run a number of experiments on the 3D representations of teeth. For example, the mesh element features have been provided to a representation generation neural network which is based on a U-Net model, and also to a representation generation model based on a variational autoencoder with continuous normalizing flows. Based on experiments, it was found that systems using a full complement of the mesh element features (e.g., “XYZ” coordinates tuple, “Normal vector”, “Vertex Curvature”, Points-Pivoted, and Normals-Pivoted) were at least 3% more accurate than systems that did not.

Points-Pivoted describes “XYZ” coordinates tuples that have local coordinate systems (e.g., at a centroid of tooth). Normals-Pivoted describes “Normal Vectors” which have local coordinate systems (e.g., at the centroid of the tooth). Furthermore, training converges more quickly when the full complement of the mesh element features are used. Stated another way, the machine learning models trained using the full complement of the mesh element features tended to be more accurate more quickly (at earlier epochs) than systems which did not. For a system which has previously been 91% accurate, an improvement in accuracy of 3% reduces an actual error rate by more than 30%.

Such feature vectors may be presented as an input of a predictive model. In some implementations, such feature vectors may be presented to one or more internal layers of a neural network which is part of one or more of those predictive models.

According to particular implementations, convolution layers in the various 3D neural networks described herein may use edge data to perform mesh convolution. The use of edge data guarantees that the 3D neural network is not sensitive to different input orders of 3D elements. In addition to or separate from using the edge data, convolution layers may use vertex data to perform the mesh convolution. The use of the vertex data is advantageous in that there are typically fewer vertices than edges or faces, so vertex-oriented processing may lead to a lower processing overhead and lower computational cost. In addition to or separate from using the edge data or the vertex data, the convolution layers may use face data to perform the mesh convolution. Furthermore, in addition to or separate from using the edge data, the vertex data, or the face data, the convolution layers may use voxel data to perform the mesh convolution. The use of the voxel data is advantageous in that, depending on the granularity chosen, there may be significantly fewer voxels to process compared to the vertices, the edges, or the faces in the mesh. Sparse processing (with the voxels) may lead to a lower processing overhead and a lower computational cost (especially in terms of computer memory or RAM usage).

Because generator networks of this disclosure can be implemented as one or more neural networks, the generator may contain an activation function. When executed, the activation function outputs a determination of whether or not a neuron in a neural network will fire (e.g., send output to a next layer). Some activation functions may include: binary step functions, or linear activation functions. Other activation functions impart non-linear behavior to the neural network, including: sigmoid/logistic activation functions, Tanh (hyperbolic tangent) functions, rectified linear units (ReLU), leaky ReLU functions, parametric ReLU functions, exponential linear units (ELU), softmax function, swish function, Gaussian error linear unit (GELU), or scaled exponential linear unit (SELU). The linear activation function may be well suited to some regression applications (among other applications), in an output layer. The sigmoid/logistic activation function may be well suited to some binary classification applications (among other applications), in an output layer. The softmax activation function may be well suited to some multiclass classification applications (among other applications), in an output layer. The sigmoid activation function may be well suited to some multilabel classification applications (among other applications), in an output layer. The ReLU activation function may be well suited in some convolutional neural network (CNN) applications (among other applications), in a hidden layer. The Tanh and/or sigmoid activation function may be well suited in some recurrent neural network (RNN) applications (among other applications), for example, in a hidden layer. There are multiple optimization algorithms which can be used in the training of the neural networks of this disclosure (such as in updating the neural network weights), including gradient descent (which determines a training gradient using first-order derivatives and is commonly used in the training of neural networks), Newton's method (which may make use of second derivatives in loss calculation to find better training directions than gradient descent, but may require calculations involving Hessian matrices), and conjugate gradient methods (which may yield faster convergence than gradient descent, but do not require the Hessian matrix calculations which may be required by Newton's method). In some implementations, additional methods may be employed to update weights, in addition to or in place of the techniques described above. These additional methods include Levenberg-Marquardt method and/or simulated annealing. The backpropagation algorithm is used to transfer the results of loss calculation back into the neural network so that neural network weights can be adjusted, and learning can progress.

Neural networks contribute to the functioning of many of the applications of the present disclosure. The neural networks of the present disclosure may embody part or all of a variety of different neural network models. Examples include the U-Net architecture, a multi-later perceptron (MLP), a transformer, a pyramid architecture, a recurrent neural network (RNN), autoencoder, a variational autoencoder, a regularized autoencoder, a conditional autoencoder, a capsule neural network, a capsule autoencoder, a stacked capsule autoencoder, a denoising autoencoder, a sparse autoencoder, a conditional autoencoder, a long/short term memory (LSTM), a gated recurrent unit (GRU), a deep belief network (DBN), a deep convolutional network (DCN), a deep convolutional inverse graphics network (DCIGN), a liquid state machine (LSM), an extreme learning machine (ELM), an echo state network (ESN), a deep residual network (DRN), a Kohonen network (KN), a neural Turing machine (NTM), or a generative adversarial network (GAN). In some implementations, an encoder structure or a decoder structure may be used. Each of these models provides one or more of its own particular advantages. For example, a particular neural network architecture may be especially well suited to a particular ML technique. For example, autoencoders are particularly suited for the classification of 3D clinical data, due to an ability to convert the 3D clinical data into a form which is more easily classifiable.

In some implementations, the neural networks of this disclosure can be adapted to operate on the 3D point cloud data (alternatively on the 3D meshes or the 3D voxelized representation). Numerous neural network implementations may be applied to the processing of the 3D representations and may be applied to training predictive and/or generative models for clinical applications, including: PointNet, PointNet++, SO-Net, spherical convolutions, Monte Carlo convolutions and dynamic graph networks, PointCNN, ResNet, MeshNet, DGCNN, VoxNet, 3D-ShapeNets, Kd-Net, Point GCN, Grid-GCN, KCNet, PD-Flow, PU-Flow, MeshCNN and DSG-Net.

Some implementations of the techniques of this disclosure incorporate the use of the autoencoder. The autoencoders that can be used in accordance with aspects of this disclosure include but are not limited to: AtlasNet, FoldingNet and 3D-PointCapsNet. Some autoencoders may be implemented based on PointNet.

Representation learning may be applied to techniques of this disclosure by training a neural network to learn a representation of the clinical data, and then using another neural network to classify the representation. Some implementations may use a VAE or a Capsule Autoencoder to generate a representation of reconstruction characteristics of one or more 3D representations of the clinical data (e.g., of wounds or appendages). Then that representation (either a latent vector or a latent capsule) may be used as input to a classification module.

Systems of this disclosure may implement end-to-end training. Some of the end-to-end training-based techniques of this disclosure may involve two or more neural networks, where the two or more neural networks are trained together (i.e., the weights are updated concurrently during the processing of each batch of input clinical data). End-to-end training may, in some implementations, be applied to the classification techniques described herein.

According to some of the transfer learning-based implementations of this disclosure, a neural network (e.g., a U-Net) may be trained on a first task. The neural network trained on the first task may be executed to provide one or more of the starting neural network weights for training of another neural network that is trained to perform a second task. The first neural network may learn the low-level neural network features of the clinical data and be shown to work well at the first task. The second neural network may exhibit faster training and/or improved performance by using the first neural network as a starting point in training. Certain layers may be trained to encode neural network features for the clinical data that were in the training dataset. These layers may thereafter be fixed (or be subjected to minor changes over the course of training) and be combined with other neural network components, such as additional layers, which are trained for other tasks. In this manner, a portion of a neural network for one or more of the techniques of the present disclosure may receive initial training on another task, which may yield important learning in the trained network layers. This encoded learning may then be built upon with further task-specific training of another network.

In some implementations, a neural network trained to output predictions based on the clinical data may first be partially trained on one of the following publicly available datasets, before being further trained on the clinical data: Google PartNet dataset, ShapeNet dataset, ShapeNetCore dataset, Princeton Shape Benchmark dataset, ModelNet dataset, ObjectNet3D dataset, Thingi10K dataset (which is especially relevant to 3D printed parts validation), ABC: A Big CAD Model Dataset For Geometric Deep Learning, ScanObjectNN, VOCASET, 3D-FUTURE, MCB: Mechanical Components Benchmark, PoseNet dataset, PointCNN dataset, MeshNet dataset, MeshCNN dataset, PointNet++ dataset, PointNet dataset, or PointCNN dataset.

Transfer learning may be employed to further train any of the following networks: GCN (Graph Convolutional Networks), PointNet, ResNet or any of the other neural networks from the published literature which are listed above.

Systems of this disclosure may train ML models with the representation learning. The advantages of the representation learning include the fact that a generative network is guaranteed to receive input data with a known size and/or standard format, as opposed to receiving input with a variable size or structure. The representation learning may produce improved performance over other methods, since noise in the input data may be reduced (e.g., since a representation generation model extracts the important aspects of an inputted representation (e.g., a mesh or a point cloud) through loss calculations or network architectures chosen for that purpose). Such loss calculation methods may include a KL-divergence loss, a reconstruction loss or other losses disclosed herein. The representation learning may reduce a size of a dataset required for training a model, since the representation model learns the representation, enabling the generative network to focus on learning the generative task. The result may be improved model generalization because meaningful features of the input data (e.g., local and/or global features) are made available to the generative network. In some instances, the transfer learning may first train the representation generation model. That representation generation model (in whole or in part) may then be used to pre-train a subsequent model, such as a classification model. The representation generation model may benefit from taking mesh element features as input, to improve the understanding of the structure and/or the shape of the 3D clinical data in the training dataset.

One or more of the neural networks models of this disclosure may have attention gates integrated within. The attention gate integration provides the enhancement of enabling the associated neural network architecture to focus resources on one or more input values. In some implementations, an attention gate may be integrated with a U-Net architecture, with the advantage of enabling the U-Net to focus on certain inputs. The attention gate may also be integrated with an encoder or with an autoencoder to improve resource efficiency, in accordance with aspects of this disclosure.

The mesh comparison module may compare two or more meshes, for example, for the computation of a loss function or for the computation of a reconstruction error. Some implementations may involve a comparison of a volume and/or an area of the two meshes. Some implementations may involve computation of a minimum distance between corresponding vertices/faces/edges/voxels of the two meshes. For a point in one mesh (vertex point, mid-point on edge, or triangle center, for example) compute the minimum distance between that point and the corresponding point in the other mesh. In the case that the other mesh has a different number of elements or there is otherwise no clear mapping between corresponding points for the two meshes, different approaches can be considered. For example, the open-source software packages CloudCompare and MeshLab each have mesh comparison tools which may play a role in the mesh comparison module for the present disclosure. In some implementations, a Hausdorff Distance may be computed to quantify the difference in shape between two meshes. The open-source software tool Metro, developed by the Visual Computing Lab, can also play a role in quantifying the difference between two meshes. The following paper describes the approach taken by Metro, which may be adapted by the neural networks applications of the present disclosure for use in mesh comparison and difference quantification: “Metro: measuring error on simplified surfaces” by P. Cignoni, C. Rocchini and R. Scopigno, Computer Graphics Forum, Blackwell Publishers, vol. 17(2), June 1998, pp 167-174.

Some techniques of this disclosure may incorporate the operation of, for one or more points on a first mesh, shooting a ray normal to the mesh surface and calculating the distance before that ray is incident upon a second mesh. The lengths of the resulting line segments may be used to quantify the distance between the first and second meshes. According to some techniques of this disclosure, the distance may be assigned a color based on the magnitude of that distance and that color may be applied to the first mesh, by way of visualization.

An autoencoder, such as a variational autoencoder (VAE), may be trained to encode 3D clinical data in a latent space vector A, which may exist in an information-rich low-dimensional latent space. This latent space vector A may be particularly suitable for later processing by the techniques disclosed herein, because latent space vector A enables complex 3D clinical data (e.g., a 3D mesh comprising thousands of mesh elements) to be efficiently manipulated. Such a VAE may be trained to reconstruct the latent space vector A back into a facsimile of the input mesh. In some implementations, the latent space vector A may be strategically modified, so as to result in changes to the reconstructed mesh. The term mesh should be considered in a non-limiting sense to be inclusive of a 3D mesh, a 3D point cloud, and a 3D voxelized representation.

The 3D representation reconstruction VAE may advantageously make use of loss functions, nonlinearities (aka neural network activation functions) and/or solvers which are not mentioned by existing techniques. Examples of loss functions may include: mean absolute error (MAE), mean squared error (MSE), L1-loss, L2-loss, KL-divergence, entropy, and reconstruction loss. Such loss functions enable each generated prediction to be compared against the corresponding ground truth value in a quantified manner, leading to one or more loss values which can be used to train, at least in part, one or more of the neural networks. Examples of solvers may include: dopri5, bdf, rk4, midpoint, adams, explicit_adams, and fixed_adams. The solvers may enable the neural networks to solve systems of equations and corresponding unknown variables. Examples of nonlinearities may include: tanh, relu, softplus, elu, swish, square, and identity. The activation functions may be used to introduce nonlinear behavior to the neural networks in a manner that enables the neural networks to better represent the training data. The losses may be computed through the process of training the neural networks via backpropagation. Neural network layers such as the following may be used: ignore, concat, concat_v2, squash, concatsquash, scale, and concatscale.

Reconstruction loss may compare a predicted output to a ground truth (or reference) output. Systems of this disclosure may compute reconstruction loss as a combination of the L1 loss and the MSE loss, as shown in the following line of pseudocode: reconstruction_loss=0.5*L1 (all_points_target,all_points_predicted)+0.5*MSE (all_points_target,all_points_predicted). In the above example, all_points_target is a 3D representation (e.g., a 3D mesh or a point cloud) corresponding to ground truth data (e.g., a ground truth example of 3D clinical data). In the above example, all_points_predicted is a 3D representation (e.g., a 3D mesh or a point cloud) corresponding to generated or predicted data (e.g., a generated example of 3D clinical data). Other implementations of reconstruction loss may additionally (or alternatively) involve L2 loss, mean absolute error (MAE) loss or Huber loss terms.

Reconstruction error may compare reconstructed output data (e.g., as generated by a reconstruction autoencoder) to the original input data (e.g., the data which were provided to the input of the reconstruction autoencoder). Systems of this disclosure may compute reconstruction error as a combination of the L1 loss and the MSE loss, as shown in the following line of pseudocode: reconstruction_error=0.5*L1 (all_points_input, all_points_reconstructed)+0.5*MSE (all_points_input, all_points_reconstructed). In the above example, all_points_input is a 3D representation (e.g., a 3D mesh or a point cloud) corresponding to input data (e.g., the 3D clinical data which is provided to the input of an ML model). In the above example, all_points_reconstructed is a 3D representation (e.g., a 3D mesh or a point cloud) corresponding to reconstructed (or generated) data (e.g., generated 3D clinical data).

In other words, the reconstruction loss is concerned with computing a difference between a predicted output and a reference output, whereas the reconstruction error is concerned with computing a difference between a reconstructed output and an original input from which the reconstructed data are derived.

In some implementations, the 3D representation reconstruction autoencoder (e.g., a reconstruction VAE model for reconstructing 3D clinical data) may be trained on examples of the 3D clinical data. FIG. 4 shows a method of training such a reconstruction autoencoder, which may be used by the first ML module 304 to generate representations (i.e., the latent representations 305). According to the reconstruction autoencoder training shown in FIG. 4, the reconstruction loss 408 may be computed between the reconstructed output (i.e., the second 3D representation 406) and the ground truth (i.e., the first 3D representation 108), using the loss calculation methods described herein (e.g., the reconstruction loss, or the KL-Divergence loss, among others). Backpropagation may be used to train the encoder 402 and the decoder 404, at least in part, using the reconstruction loss 408.

The reconstruction autoencoder in FIG. 4, of which the reconstruction VAE model is an example, may be trained to encode the clinical data 101 as a reduced-dimensionality form, called a latent space vector (i.e., the latent representations 305). The clinical data 101 may be provided to the encoder 402, encoded into the latent space vector, and then reconstructed into a facsimile of the input mesh (i.e., the second 3D representation 406) using the decoder 404. One advantage of this process is that the encoder 402 may become trained to convert the clinical data 101 (or mesh of aspects of the body of the patient) into a reduced-dimension form that can be used in the training and deployment of the classification techniques of this disclosure. This reduced-dimensionality form of the clinical data 101 may enable the second ML module 306 (e.g., a classification module) shown in FIG. 3 to more efficiently encode the reconstruction characteristics of the clinical data 101, and better learn to classify the clinical data 101, thereby providing technical improvements in terms of both data precision and resource footprint.

A reconstructed mesh (i.e., the second 3D representation 406) may be compared to an input mesh (i.e., the first 3D representation 108), for example, using the reconstruction error (as described herein), which quantifies the differences between the reconstructed and input meshes. This reconstruction error may, in some implementations, be computed using Euclidean distances between corresponding mesh elements between the two meshes (i.e., the reconstructed and input meshes). There are other methods of computing this error which may be described elsewhere in this disclosure.

In some implementations, the 3D representations which are provided to the reconstruction VAE may first be rearranged into lists of mesh elements (e.g., a 3D mesh may be rearranged into lists of vertices, a 3D point cloud may be rearranged into lists of points, etc.) before being provided to the encoder 402.

The performance of the reconstruction VAE may, in some implementations, be measured using reconstruction error calculations. In some examples, the reconstruction error may be computed as element-to-element distances between the two meshes, for example, using Euclidean distances. Other distance measures are possible in accordance with various implementations of the techniques of this disclosure, such as Cosine distance, Manhattan distance, Minkowski distance, Chebyshev distance, Jaccard distance (e.g., intersection over union of meshes), Haversine distance (e.g., distance across a surface), and Sorensen-Dice distance.

The performance of the reconstruction VAE may, in some implementations, be verified via reconstruction error plots and/or other key performance indicators.

Autoencoders of this disclosure (such as VAE or capsule autoencoders) may be trained on other types of data (e.g., 2D images, text data, categorical data, spatiotemporal data, real-time data and/or vectors of real numbers). Such autoencoders may be provisioned to reconstruct examples of those other types of data. Data may be qualitative or quantitative. Data may be nominal or ordinal. Data may be discrete or continuous. Data may be structured, unstructured or semi-structured. The autoencoders of this disclosure may convert such data into latent representations (e.g., latent vectors or latent capsules) for classification by the second ML module 306 shown in FIG. 3.

Techniques of this disclosure (e.g., the first ML module 304 shown in FIG. 3) may, in some implementations, use PointNet, PointNet++, or derivative neural networks (e.g., networks trained via transfer learning using either PointNet or PointNet++ as a basis for training) to extract local or global neural network features from a 3D point cloud or other 3D representation (e.g., a 3D point cloud describing aspects of the body of the patient-such as the appendage or the skin). Techniques of this disclosure (e.g., the first ML module 304) may, in some implementations, use U-Nets to extract hierarchical neural network features (e.g., local, intermediate, or global neural network features) from the 3D point cloud or the other 3D representation.

3D clinical data, as used herein, is intended to be used in a non-limiting fashion to encompass any representations of 3-dimensions or higher orders of dimensionality (e.g., 4D, 5D, etc.), and it should be appreciated that ML models can be trained using the techniques disclosed herein to operate on representations of higher orders of dimensionality.

A U-Net may comprise an encoder, followed by a decoder. An architecture of the U-Net may resemble a U shape. The encoder may be trained to extract one or more global neural network features from an input 3D representation, zero or more intermediate-level neural network features, or one or more local neural network features (at the most local level as contrasted with the most global level). The output from each level of the encoder may be passed along to an input of corresponding levels of a decoder (e.g., by way of skip connections). Like the encoder, the decoder may operate on multiple levels of global-to-local neural network features. For instance, the decoder may output a representation of the input data which may contain global, intermediate, or local information about the input data. The U-Net may, in some implementations, generate an information-rich (optionally reduced-dimensionality) representation of the input data, which may be more easily consumed by other generative or discriminative machine learning models.

A transformer may be trained to use self-attention to generate, at least in part, representations of a 3D clinical data. A transformer may encode long-range dependencies (e.g., encode relationships between a large number of inputs). The transformer may also comprise an encoder or a decoder. The encoder may, in some implementations, operate in a bi-directional fashion or may operate a self-attention mechanism. The decoder may, in some implementations, may operate a masked self-attention mechanism, may operate a cross-attention mechanism, or may operate in an auto-regressive manner. The self-attention operations of the transformers described herein may, in some implementations, relate different positions or aspects of an individual example of the 3D clinical data in order to compute a reduced-dimensionality representation of that 3D clinical data. The cross-attention operations of the transformers described herein may, in some implementations, mix or combine aspects of two (or more) different 3D clinical data. The auto-regressive operations of the transformers described herein may, in some implementations, consume previously generated aspects of the 3D clinical data as additional input when generating a new or modified 3D clinical data. Either a transformer encoder or a transformer decoder may, in some implementations, generate a latent form of the 3D clinical data, which may be used as an information-rich reduced-dimensionality representation of the 3D clinical data, which may be more easily consumed by other generative or discriminative machine learning models (e.g., the second ML module 306 shown in FIG. 3).

Techniques of this disclosure may, in some instances, be trained using federated learning. The federated learning may enable multiple remote clinicians to iteratively improve a machine learning model (e.g., either or both of the first ML module 304 and the second ML module 306), while protecting data privacy (e.g., the clinical data 101 may not need to be sent “over the wire” to a third party). Data privacy is particularly important to the clinical data 101, which is protected by applicable laws. A clinician may receive a copy of a machine learning model, use a local machine learning program to further train that ML model using locally available data from the local clinic, and then send the updated ML model back to a central hub or third party. The central hub or third party may integrate the updated ML models from multiple clinicians into a single updated ML model which benefits from the learnings of recently collected patient data at the various clinical sites. In this way, a new ML model may be trained which benefits from additional and updated patient data (possibly from multiple clinical sites), while those patient data are never actually sent to the 3rd party. Training on a local in-clinic device may, in some instances, be performed when the device is idle or otherwise be performed during off-hours (e.g., when the patients are not being treated in the clinic). Devices in the clinical environment for the collection of data and/or the training of ML models for techniques described here may include smart phones equipped with stereophotographic cameras, smart phones equipped with single cameras which run software that enables stereophotographic operation by the capture of images from multiple views, other hand-held devices, 3D scanners, intra-oral scanners, CT scanners, X-ray machines, laptop computers, servers, or desktop computers. In addition to the federated learning techniques, in some implementations, contrastive learning may be used to train, at least in part, the ML models described herein. Contrastive learning may, in some instances, augment samples in a training dataset to accentuate the differences in samples from difference classes and/or increase the similarity of samples of the same class, which may improve the accuracy of the classification techniques described herein.

Unless otherwise indicated, all numbers expressing feature sizes, amounts, and physical properties used in the specification and claims are to be understood as being modified by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific implementations have been illustrated and described herein, it will be appreciated by those of ordinary skill in the art that a variety of alternate and/or equivalent implementations can be substituted for the specific implementations shown and described without departing from the scope of the present disclosure. This application is intended to cover any adaptations or variations of the specific implementations discussed herein. Therefore, it is intended that this disclosure be limited only by the claims and the equivalents thereof.

1. A method for clinical data analysis, the method comprising:
receiving a first three-dimensional (3D) representation that is representative of clinical data, wherein the first 3D representation comprises one or more mesh elements;
computing one or more mesh element features for the one or more mesh elements;
providing the one or more mesh element features as an input to a first machine learning (ML) module; and
executing the first ML module to encode the first 3D representation into one or more latent representations;
providing the one or more latent representations to a second ML module that is different from the first ML module; and
executing the second ML module to classify the clinical data represented in the first 3D representation into at least one predicted classification label.

2. The method of claim 1, further comprising:
receiving at least one ground truth classification label for the first 3D representation;
computing a loss that quantifies a difference between the at least one ground truth classification label and the at least one predicted classification label; and
using the loss to train the second ML module.

3. The method of claim 1, wherein the clinical data is representative of a skin of a patient, and wherein the second ML module is configured to classify the clinical data into at least one of a type of a skin abnormality, a current state of the skin abnormality, a future state of the skin abnormality, a current state of an implant site including an implant on the skin, and a future state of the implant site on the skin.

4. The method of claim 3, wherein the type of skin abnormality is at least one of a tumor, a wound, a burn, a rash, a puncture, a cyst, an infection, a skin growth, a bruise, a cut, a tear, an abrasion, a scratch, an ulcer, and a laceration.

5. The method of claim 3, wherein the implant is at least one of a skin graft and a device implant.

6. The method of claim 1, wherein the clinical data is representative of an appendage, and wherein the second ML module is configured to classify the clinical data into a current state of a swelling in the appendage or a future state of the swelling in the appendage.

7. The method of claim 1, wherein the clinical data is representative of a torso, and wherein the second ML module is configured to classify the clinical data into a current state of a swelling in the torso or a future state of the swelling in the torso.

8. The method of claim 1, wherein the clinical data is representative of an article disposed on a skin of a patient, and wherein the second ML module is configured to classify the clinical data into a current state of the article.

9. The method of claim 8, wherein the article is a wrapping on the skin of the patient, and wherein the current state of the article includes a fit of the wrapping on the skin of the patient.

10. The method of claim 1, further comprising executing the first ML module to reconstruct the one or more latent representations into a second 3D representation that is a facsimile of the first 3D representation.

11. The method of claim 10, further comprising computing a reconstruction error that quantifies a difference between the first 3D representation and the second 3D representation.

12. The method of claim 11, further comprising:
determining at least one region of the first 3D representation that has the reconstruction error greater than a predetermined threshold; and
determining that the at least one region corresponds to at least one of a skin abnormality and an article disposed on a skin of a patient.

13. The method of claim 1, wherein at least one of the one or more mesh elements has at least one associated meta data value.

14. The method of claim 13, wherein the at least one associated meta data value comprises data pertaining to at least one of a color of an object, a temperature of the object, and a surface impedance of the object.

15. (canceled)

16. The method of claim 10, wherein the first ML module is an autoencoder neural network.

17. The method of claim 16, wherein an encoder of the autoencoder neural network is configured to encode the first 3D representation into the one or more latent representations.

18. The method of claim 17, wherein a decoder of the autoencoder neural network is configured to reconstruct the one or more latent representations into the second 3D representation.

19. The method of claim 18, further comprising:
computing a reconstruction loss that quantifies a difference between the first 3D representation and the second 3D representation; and
using the reconstruction loss to train the first ML module.

20. The method of claim 18, wherein the decoder of the autoencoder neural network is remotely located from the encoder.

21. The method of claim 16, wherein the autoencoder neural network comprises a variational autoencoder (VAE) neural network.

22. The method of claim 1, wherein executing the first ML module to encode the first 3D representation into the one or more latent representations comprises executing the first ML module, by a first processor, to encode the first 3D representation into the one or more latent representations, and wherein executing the second ML module to classify the clinical data represented in the first 3D representation into the at least one predicted classification label comprises executing, by a second processor that is different from the first processor, to classify the clinical data represented in the first 3D representation into the at least one predicted classification label.

23. A computing device comprising:
an interface configured to receive a first three-dimensional (3D) representation that is representative of clinical data, wherein the first 3D representation comprises one or more mesh elements;
a memory communicably coupled to the interface and configured to store the first 3D representation; and
a processor communicably coupled to the interface and the memory, the processor configured to:
compute one or more mesh element features for the one or more mesh elements;
provide the one or more mesh element features as an input to a first machine learning (ML) module;
execute the first ML module to encode the first 3D representation into one or more latent representations;
provide the one or more latent representations to a second ML module that is different from the first ML module; and
execute the second ML module to classify the clinical data represented in the first 3D representation into at least one predicted classification label.

24. (canceled)

25. (canceled)

26. A method for detecting an anomaly, the method comprising:
receiving a first three-dimensional (3D) representation that is representative of clinical data;
providing the first 3D representation as an input to a first machine learning (ML) module;
executing the first ML module to:
encode the first 3D representation into one or more latent representations; and
reconstruct the one or more latent representations into a second 3D representation that is a facsimile of the first 3D representation;
computing a reconstruction error that quantifies a difference between the first 3D representation and the second 3D representation;
determining at least one region of the first 3D representation that has the reconstruction error greater than a predetermined threshold; and
determining that the at least one region corresponds to the anomaly.

27.-35. (canceled)

36. A method for detecting a swelling, the method comprising:
receiving a first three-dimensional (3D) representation that is representative of clinical data, wherein the clinical data is representative of a skin of a patient or an appendage of the patient;
providing the first 3D representation as an input to a first machine learning (ML) module;
executing the first ML module to encode the first 3D representation into one or more latent representations;
providing the one or more latent representations to a second ML module that is different from the first ML module; and
executing the second ML module to classify the clinical data into a current state of the swelling or a future state of the swelling.

37. The method of claim 36, wherein the current state of the swelling or the future state of the swelling is used to detect lymphedema.

38. The method of claim 36, wherein:
receiving the first 3D representation comprises receiving at least two first 3D representations that are representative of the clinical data obtained at different time intervals;
providing the first 3D representation as the input to the first ML module comprises providing the at least two first 3D representations as the input to the first ML module;
executing the first ML module to encode the first 3D representation into the one or more latent representations comprises executing the first ML module to encode the at least two first 3D representations into corresponding one or more latent representations;
providing the one or more latent representations to the second ML module comprises providing the corresponding one or more latent representations to the second ML module; and
executing the second ML module to classify the clinical data into the current state of the swelling or the future state of the swelling comprises comparing the corresponding one or more latent representations by the second ML module to classify the clinical data into the current state of the swelling or the future state of the swelling.
